(19) United States
(12) Patent Application Publication
(10) Pub. No.: US 2019/0264209 A1
(43) Pub. Date: Aug. 29, 2019

(54) SMALL VIRAL RNA MOLECULES AND USES THEREOF

(71) Applicant: The University of Queensland, St. Lucia (AU)

(72) Inventors: Peer Schenk, Tennyson (AU); Shazia Iram, Yeronga (AU)

(73) Assignee: The University of Queensland, St. Lucia (AU)

(21) Appl. No.: 16/234,165

(22) Filed: Dec. 27, 2018

Related U.S. Application Data
(63) Continuation of application No. 13/879,491, filed on Nov. 19, 2013, now abandoned.

(30) Foreign Application Priority Data
Oct. 15, 2010 (AU) 2010904623

(51) Int. Cl. C12N 15/113; C12N 15/82

(52) U.S. Cl. CPC C12N 15/1131; C12N 15/8283; C12N 15/8218

(57) ABSTRACT

The present invention discloses small plant virus RNA molecules involved in modulating a plant defence response, particularly non-translated, plant viral microRNA molecules. The present invention also provides methods of their production and uses of these microRNA molecules for reducing a susceptibility of a plant to a pathogen.

### FIELD OF THE INVENTION

THIS INVENTION relates to plant molecular biology and particularly RNA molecules. More particularly, this invention relates to non-translated, small plant viral RNA molecules capable of modulating a plant defence response, methods of their production and uses thereof.

### BACKGROUND OF THE INVENTION

After invasion of their host, plant viruses encounter a comprehensive arsenal of defence mechanisms (Soosaar et al., 2005), including virus-specific RNA interference (RNAi), induction of programmed cell death and activation of plant defence genes. To counteract the host RNAi response, viruses have evolved a myriad of suppressors of gene silencing that act at different stages in RNAi pathways (Azevedo et al., 2010). RNAi suppressor proteins are encoded by both plant (Kasschau & Carrington, 1998) and animal viruses (Haasnoot et al., 2007; Li et al., 2004) and non-coding adenovirus RNAs have been shown to act as a suppressor of gene silencing in human cell lines (Lu & Cullen, 2004).

Following the first report of virus-encoded microRNAs (miRNAs) from the Epstein-Barr virus in 2004 (Pfeffer et al., 2004), more than 142 microRNAs have now been identified from 15 vertebrate viruses and one insect virus (Hussain et al., 2008). Some animal viral microRNAs target host genes that promote immune response genes or apoptosis (Stern-Ginossar et al., 2007; Choy et al., 2008), and others act as controlling molecules in viral gene expression and replication (Murphy et al., 2008). A recent study has shown that an Influenza RNA virus, engineered for production of a cellular microRNA miR-124, is capable of producing functional microRNAs without any effect on virus replication (Varble et al., 2010).

### SUMMARY OF THE INVENTION

The present invention has arisen from the inventors' unexpected discovery of a new class of small plant virus RNA molecules involved in modulating a plant defence response that are distinguishable from any previously identified class of small virus encoded RNA molecules.

In a first aspect, the invention provides an isolated plant viral RNA molecule that comprises a nucleotide sequence comprising no more than 30 contiguous nucleotides, which nucleotide sequence is capable of modulating a plant defence response.

Suitably, said isolated plant viral RNA molecule comprises a nucleotide sequence that is capable of modulating the expression and/or activity of one or more plant defence nucleic acids. Typically, said isolated plant viral RNA molecule comprises a nucleotide sequence that is capable of at least partially reducing, mitigating, silencing, or otherwise decreasing the expression and/or activity of one or more plant defence nucleic acids.

In one embodiment, said plant defence nucleic acid is a viral defence nucleic acid.

In one preferred form, said isolated RNA molecule is encoded by a genome of an RNA virus. For example, said isolated RNA molecule is encoded by the genome of a positive sense single-stranded RNA virus, a negative sense single-stranded RNA virus or a double-stranded RNA virus. Suitably, said isolated RNA molecule is encoded by a genome of a virus of the Potyviridae, Virgaviridae, Bunyaviridae, or Reoviridae families. Suitably, said isolated RNA molecule is encoded by the genome of a virus of the genus Potyvirus, Tobamovirus, Tospovirus, or Fijivirus. Suitably, said isolated RNA molecule is encoded by the genome of a virus of a species of Turnip mosaic virus, Tobacco mosaic virus, Tomato spotted wilt virus, or Fiji disease virus.

In one preferred form, the plant is selected from the group consisting of a monocot and a dicot. Typically, although not exclusively, said plant is selected from the group consisting of Arabidopsis, corn, wheat, rice, barley, oats, sugarcane, sunflower, tobacco, Nicotiana, cotton, soy, tomato, canola, and alfalfa.

Non-limiting examples of the isolated plant viral RNA molecules of the invention are set forth in SEQ ID NOs: 1-82 and their complements SEQ ID NOS: 139-220, respectively (Table 1).

This aspect of the invention also provides a modified, isolated plant viral RNA molecule, a precursor of the isolated plant viral RNA molecule, a fragment of the isolated plant viral RNA molecule and/or an RNA or DNA molecule at least partly complementary to said isolated plant viral RNA molecule.

In a second aspect, the invention provides a method of producing the isolated RNA molecule of the first aspect, said method including the step of isolating one or more of said isolated RNA molecules from a nucleic acid sample obtained from a plant pathogen or a plant infected with said plant pathogen.

In one preferred form, said plant pathogen is a virus. Preferably, said plant pathogen is an RNA virus. Suitably, said plant pathogen is a positive sense single-stranded RNA virus, a negative sense single-stranded RNA virus or a double-stranded RNA virus. Suitably, said plant pathogen is a virus of the family Potyviridae, Virgaviridae, Bunyaviridae, or Reoviridae. Suitably, said plant pathogen is a virus of the genus Potyvirus, Tobamovirus, Tospovirus, or Fijivirus. Suitably, said plant pathogen is a virus of a species of Turnip mosaic virus, Tobacco mosaic virus, Tomato spotted wilt virus, or Fiji disease virus.

In a third aspect, the invention provides a genetic construct which comprises one or a plurality of the isolated RNA molecules according to the first aspect.

In one particular embodiment, the genetic construct is an expression construct comprising a DNA sequence complementary to one or a plurality of the isolated RNA molecules of the first aspect operably linked or connected to one or more additional nucleotide sequences.

In a fourth aspect, the invention provides a host cell comprising the genetic construct of the third aspect.

In an fifth aspect, the invention provides a method of identifying a plant defence nucleic acid, said method including the step of identifying a plant defence nucleic acid that is modulated by (i) the isolated RNA molecule of the first aspect, or (ii) the isolated RNA molecule produced according to the method of the second aspect.

Suitably, the expression and/or activity of the plant defence nucleic acid is modulated by the isolated RNA molecule. Preferably, the expression and/or activity of the plant defence nucleic acid is at least partly reduced, lowered or otherwise decreased by the isolated RNA molecule.

In a sixth aspect, the invention provides a method of modifying a plant defence nucleic acid, said method including the step of modifying a nucleotide sequence of the plant defence nucleic acid to be at least partially resistant to modulation by the isolated RNA molecule of the first aspect.

Preferably, said plant defence nucleic acid is modified by mutating a region that the isolated RNA molecule of the first aspect binds, anneals to, hybridises to, or otherwise recognises. Suitably, said plant defence nucleic acid is modified by a nucleotide sequence deletion, insertion, and/or substitution. Preferably, said plant defence nucleic acid is modified by introducing a silent mutation.

In one particular embodiment, said plant defence nucleic acid is modified by zinc finger gene targeting.

In a seventh aspect, the invention provides an isolated modified plant defence nucleic acid, which isolated plant defence nucleic acid has been modified using the method of the sixth aspect.

In a eighth aspect, the invention provides a method of reducing a susceptibility of a plant to a pathogen, said method including the step of introducing the isolated modified plant defence nucleic acid of the seventh aspect into said plant to thereby reduce, decrease, or mitigate the susceptibility of said plant to said pathogen.

In a ninth aspect, the invention provides a plant or a plant cell comprising the isolated modified plant defence nucleic acid of the seventh aspect.

In a tenth aspect, the invention provides a method of reducing a susceptibility of a plant population to a pathogen, said method including the step of selecting for at least one plant that comprises a naturally occurring plant defence nucleic acid that is not susceptible to modulation by the isolated RNA molecule of the first aspect, or the isolated RNA molecule produced according to the method of the second aspect, which thereby has a reduced, decreased, or mitigated susceptibility to said pathogen, and using the at least one plant in plant breeding.

In an eleventh aspect, the invention provides a method of reducing a susceptibility of a plant population to a pathogen, said method including the step of introducing a decoy target sequence into the plant to thereby reduce, decrease, or mitigate the susceptibility of the plant to the pathogen, wherein the decoy target sequence binds, anneals to, hybridises to, or otherwise recognises and captures the isolated RNA molecule of the first aspect, or the isolated RNA molecule produced according to the method of the second aspect.

Suitably, said plant defence nucleic acid is an HVA22d nucleic acid comprising a silent mutation, which silent mutation is absent in a wild-type counterpart.

In one preferred form, said pathogen is a virus. Preferably, said pathogen is an RNA virus. Suitably, said pathogen is a virus of the family Potyviridae, Virgaviridae, Bunyaviridae, or Reoviridae. Suitable, said pathogen is a virus of the genus Potyvirus, Tobamovirus, Tospovirus, or Fijivirus. Suitably, said pathogen is a virus of a species of Turnip mosaic virus, Tobacco mosaic virus, Tomato spotted wilt virus, or Fiji disease virus.

In a twelfth aspect, the invention provides a computer-readable storage medium or device encoded with nucleotide sequence data of each of a plurality of the isolated RNA molecules according to the first aspect, and/or the isolated plant viral RNA molecules produced according to the method of the second aspect.

In an thirteenth aspect, the invention provides a nucleic acid array comprising a plurality of the isolated RNA molecules according to the first aspect, and/or the isolated plant viral RNA molecules produced according to the method of the second aspect, immobilised, affixed or otherwise mounted to a substrate.

In a fourteenth aspect, the invention provides an antibody which binds the isolated RNA molecule of the first aspect, and/or the isolated plant viral RNA molecule produced according to the method of the second aspect.

In an fifteenth aspect, the invention provides a kit comprising one or more of the isolated RNA molecules according to the first aspect, and/or the isolated plant viral RNA molecule produced according to the method of the second aspect, the antibody of the fourteenth aspect, and one or more detection reagents.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE DRAWINGS

FIGS. 1A-1D. Nuclear localisation of virus and prediction of viral microRNAs.

FIG. 1A. Isolated nuclei from Arabidopsis thaliana virus-infected cells, using oil emulsion microscopy at 100× and stained with DAPI at 40× resolution (background colour was modified for better visualisation).

FIG. 1B. Northern blot hybridisation using 800 bp coat protein (CP) DNA probe shows the presence of the replicative form of TuMV virus in the nucleus while the plus strand ssRNA of TuMV largely accumulated in the cytoplasm.

M14=Mock at 14 days post inoculation (dpi), V9=Virus-infected at 9 dpi, V14=Virus-infected at 14 dpi, Col-0=wild-type (WT) Arabidopsis thaliana.

FIG. 1C. Prediction of TuMV-mir-S1 and TuMV-mir-S2 precursors using bioinformatic software.

FIG. 1D. Predicted TuMV-mir-S1 and TuMV-mir-S2 mature microRNAs; the upper strands are the predicted guide strands of the mature microRNAs. The upper strand sequence of TuMV-mir-S1 is SEQ ID NO: 1 and the lower strand sequence is SEQ DI NO: 221. The upper strand sequence of TuMV-mir-52 is SEQ ID NO: 223 and the lower strand sequence is SEQ DI NO: 224.

FIGS. 2A-2C. Detection of mature microRNAs in Col-0 and microRNA precursors in the dcl2 dcl3 dcl4 triple mutant along with comparison of virus level in both WT and mutant background.

FIG. 2A. TuMV-mir-S1 mature microRNA and precursor microRNA in wild-type Col-0 and dcl2 dcl3 dcl4 plants, respectively.

FIG. 2B. TuMV-mir-S2 mature microRNA and precursor microRNA in wild-type Col-0 and dcl2 dcl3 dcl4 plants, respectively.

FIG. 2C. Comparison of full length viral RNA accumulation in wild-type Col-0 and dcl2 dcl3 dcl4 plants

FIGS. 3A-3D. Comparison of microRNA levels in wild-type Col-0, dcl1-8, dcl2-1, dcl3-1 and dcl4-2, dcl2 dcl4, ago1-25, hyl1-2, hst-15 mutant lines and viral microRNA localisation in the cell.

FIG. 3A. TuMV abundance in wild-type Col-0, dcl2-1, dcl3-1, dcl2 dcl4, dcl4-2, dcl1-8 Arabidopsis plants

FIG. 3B. TuMV-mir-S1 and TuMV-mir-S2 sense and antisense levels in wild-type Col-0, dcl2-1, dcl3-1, dcl2 dcl4, dcl4-2, dcl1-8 plants

FIG. 3C. Northern blot hybridisation with nuclear and cytoplasmic RNA fractions to validate localisation of viral microRNA. Only the cytoplasmic RNA fraction shows the presence of mature microRNA, and microRNA precursor is present in the nuclear RNA fraction. The loading control is the U6 small nuclear RNA.

FIG. 3D. Comparison of virus levels and TuMV-mir-S2 in wild-type Col-0 and ago1-25 plants

M14=Mock at 14 dpi, V9=Virus-infected at 9 dpi, V14=Virus infected at 14 dpi, Col-0=wild-type Col-0, cyt=cytoplasmic RNA, nuc=nuclear RNA.

FIGS. 4A-4D. Role of HVA22d in virus infection and its repression through TuMV-mir-S1 binding.

FIG. 4A. Two possible binding prediction for TuMV-mir-S1 with HVA22d. The lower TuMV-mir-S1 sequence strands are both SEQ ID NO: 225. The upper target sequence is SEQ DI NO: 223 and the lower target sequence is SEQ DI NO: 224.

FIG. 4B. Northern blot hybridisation showing partial silencing of GFP in GFP-HVA22d target fusion transgenic Arabidopsis plants. GFP expression is lowered in GFP-HVA22d target fusion plants at 14 days after TuMV infection.

FIG. 4C. qRT-PCR showing the expression of GFP and GFP-HVA22d transgenes in Arabidopsis plants after TuMV infection

FIG. 4D. Northern blot analysis of a homozygous hva22d T-DNA insertion mutant showing lower levels of TuMV in the absence of a functional HVA22d gene. A DNA copy of the coat protein of TuMV was used as the probe.

Col-0=wild-type Columbia, hva22d=T-DNA insertion mutant of HVA22d, M5=Mock at 5 dpi, V5=Virus-infected at 5 dpi, M9=Mock at 9 dpi, V9=Virus-infected at 9 dpi, M14=Mock at 14 dpi, V14=Virus-infected at 14 dpi.

FIG. 5. Schematic diagram for TuMV viral microRNA biogenesis.

FIGS. 6A-6C. Viral RNA detection in nuclear RNA of infected Arabidopsis thaliana.

FIG. 6A. Nuclear RNA blot was probed with PCR DNA containing both TuMV-miR-S1 and TuMV-miR-52 precursor sequence.

FIG. 6B. Nested PCR of —ive strand specific cDNA in nuclear RNA amplified 102 nt negative strand specific DNA.

FIG. 6C. Nuclear RNA of TuMV infected Arabidopsis was probed with TuMV plus strand specific oligonucleotide (22nt) probe.

Col-0=Col-0 total RNA, Col-0 nuc=Col-0 nuclear RNA fraction, M14=Mock at 14 dpi, V14=Virus-infected at 14 dpi, −=negative strand specific cDNA, +=Positive strand specific cDNA used as negative control for negative strand specific primers.

FIGS. 7A-7C. Detection of microRNA precursor in DCL2 and DCL4 double and single mutant plants and effect of HYL1 and HASTY mutation on TuMV-mir-S2 and virus level.

FIG. 7A. MicroRNA precursor detected in total RNA from dcl2 dcl4 plants using TuMV-mir-S1 as probe. The decreased virus accumulation in dcl2dcl4 plants results in low level of microRNA precursor.

FIG. 7B. Nuclear RNA fraction of dcl2 and dcl4 plants probed with TuMV-mir-S2 to confirm the presence of microRNA precursor in the nucleus.

FIG. 7C. Viral RNA level and TuMV-miR-S2 levels in hyl1-2 and hst-15 Arabidopsis mutants.

Col-0=WT Columbia, hyl1-2=HYL1 mutant, hst 15=HASTY mutant, dcl2 dcl4=DCL2 DCL4 double mutant, dcl2=DCL2 mutant dcl4=DCL4 mutant, nuc=nuclear RNA, M14=Mock at 14 dpi, V9=Virus-infected at 9 dpi, V14=Virus-infected at 14 dpi.

FIGS. 8A-8B. Cloning of HVA22d-GFP fusion and TuMV-mir-S1 over-expressing construct and transient suppression analysis in Nicotiana benthamiana leaves through agroinfiltration.

FIG. 8A. GFP-fusion construct of target gene HVA22d and 35S promoter driven over expressing construct for the viral microRNA precursor.

FIG. 8B. Transient GFP-fusion analysis demonstrating reduced expression of GFP as a result of co-infiltration of both GFP-HVA22d and TuMV-mir-S1precursor. 35S-GFP=GFP over-expression construct, 35S-GFP-HVA22d=GFP-HVA22d fusion construct, TuMV-mir-S1 precursor=TuMV-mir-S1 precursor over-expressing construct.

FIGS. 9A-9D. Suppression analysis of target-GFP fusion constructs and ToSWV miRNA precursors over-expression constructs in Nicotiana benthamiana leaves through agroinfiltration.

FIG. 9A. GFP-fusion analysis demonstrating reduced expression of GFP as a result of co-infiltration of both 35S-GFP-NRPD1B and ToSWV Seg L precursor.

FIG. 9B. GFP-fusion analysis demonstrating reduced expression of GFP as a result of co-infiltration of both 35S-GFP-PR5 and ToSWV Seg M 649 precursor.

FIG. 9C. GFP-fusion analysis demonstrating reduced expression of GFP as a result of co-infiltration of both 35S-GFP-BEH1 and ToSWV Seg S precursor.

FIG. 9D. GFP-fusion analysis demonstrating reduced expression of GFP as a result of co-infiltration of both 35S-GFP-EXP8 and ToSWV Seg L precursor.

FIGS. 10A-10B. Prevention of viral miRNA silencing.

FIG. 10A. HVA22D target construct with silent mutation infiltrated without the miRNA precursor construct.

FIG. 10B. HVA22D target construct with silent mutation co-infiltrated with the miRNA precursor construct.

FIGS. 11A-11C. Strategies to confer virus resistance in plants.

FIG. 11A. Wild-type plants: MicroRNA encoded by plant viruses binds and interferes with anti-viral host defence genes.

FIG. 11B. Strategy 1: A silent point mutation in the host defence gene prevents binding of microRNA leading to virus resistance.

FIG. 11C. Strategy 2: A decoy sequence with a perfect match captures microRNA from the virus leading to virus resistance.

### DETAILED DESCRIPTION OF THE INVENTION

The present invention arises from the finding of a novel class of small RNA molecules encoded by a plant virus (“plant viral miRNAs”) that are capable of modulating a plant defence response. The present inventors unexpectedly discovered that these plant viral miRNAs can be distinguished from any previously identified class of miRNAs based on their presence in plant viruses and their ability to at least partially modulate a plant host defence response.

It is to be understood that the terms “microRNA”, “viral microRNA” and “viral miRNA” refer to small plant viral RNA molecules that have the potential to target host plant genes, irrespective of the name that these molecules may be given by the scientific community.

It will be appreciated that these plant viral miRNAs exhibit different characteristics to the virus encoded miRNAs that have previously been identified in animal viruses. The present invention is based on the inventors' identification of plant viral miRNAs, the manipulation of these plant viral miRNAs, the use of plant viral miRNAs to modulate a plant defence response, and plants having reduced susceptibility to plant pathogens (e.g., viruses). The invention also concerns methods for producing novel plant viral miRNAs, use of plant viral miRNAs to (i) identify novel nucleic acid targets, and (ii) reduce a susceptibility of a plant to a pathogen, as well as arrays comprising plant viral miRNAs (“plant viral miRNA arrays”).

The term “plant” includes both plants and plant parts such as, but not limited to, plant cells, plant tissue such as leaves, stems, roots, flowers and seeds. A classification of plants may be found at http://theseedsite.co.uk/class.html.

Plants, plant cells and seeds of the invention include monocots and dicots including, but not limited to, cotton, oilseed rape, wheat, corn or maize, barley, alfalfa, peanuts, sunflowers, rice, oats, sugarcane, soybean, turf grasses, rye, sorghum, sugar cane, vegetables (e.g., chicory, lettuce, tomato, zucchini, bell pepper, eggplant, cucumber, melon, onion, and leek), tobacco, Nicotiana, potato, sugarbeet, papaya, pineapple, mango, Arabidopsis, and plants used in horticulture, floriculture or forestry (e.g., poplar, fir and eucalyptus).

As used herein, a plant that has a “reduced susceptibility” to a pathogen (e.g., a virus) is less likely to become infected by, carry and/or transmit the pathogen compared to a wild-type counterpart.

The term “nucleic acid” as used herein designates single- or double-stranded mRNA, RNA, cRNA, RNAi, miRNA and DNA inclusive of cDNA and genomic DNA. The miRNA is typically a single-stranded molecule, while the miRNA precursor is typically an at least partially self-complementary molecule capable of forming double-stranded portions (e.g., stem-loop structures). Nucleic acids may comprise naturally-occurring nucleotides or synthetic, modified or derivatised bases (e.g., inosine, methyinosine, pseudouridine, methylcytosine, etc.). Nucleic acids may also comprise chemical moieties coupled thereto to them. Examples of chemical moieties include, but are not limited to, biotin, locked nucleic acids (LNAs), peptide nucleic acids (PNAs), cholesterol, 2′ O-methyl, Morpholino, and fluorophores such as HEX, FAM, Fluorescein and FITC.

A “stem-loop structure” refers to a nucleic acid having a secondary structure that includes a region of nucleotides which are known or predicted to form a double strand (“stem portion”) that is linked on one side by predominantly single-stranded nucleotides (“loop portion”). The terms “hairpin” and “fold back” structures may also be used herein to refer to stem-loop structures. Such structures are well known in the art and these terms are used consistently with their known meanings in the art. It will be appreciated that secondary structures do not require exact base-pairing. Accordingly, the stem may include one or more base mismatches. Alternatively, the base-pairing may be exact, that is, not include any mismatches.

In one aspect, the invention provides an isolated plant viral miRNA that comprises a nucleotide sequence comprising no more than 30 contiguous nucleotides, which nucleotide sequence is capable of modulating a plant defence response.

For the purposes of this invention, by “isolated” is meant present in an environment removed from a natural state or otherwise subjected to human manipulation. Isolated material may be substantially or essentially free from components that normally accompany it in its natural state, or may be manipulated so as to be in an artificial state together with components that normally accompany it in its natural state. The term “isolated” also encompasses terms such as “enriched”, “purified”, “synthetic”, and/or “recombinant”.

The isolated plant viral miRNAs of the invention preferably have a length of from 18-30 nucleotides (nt). It should be noted that mature plant viral miRNAs typically have a length of 19-26 nucleotides, particularly 19-24 nucleotides. Accordingly, the mature miRNA may be 19 nt, 20 nt, 21 nt, 22 nt, 23 nt, or 24 nt. The plant viral miRNA may also be provided as a plant viral “miRNA precursor”, which usually has a length of 50-100 nucleotides, particularly 60-80 nucleotides. Thus, the miRNA precursor may be about 65 nt, about 70 nt or about 75 nt. It should be noted that the precursor may be produced by processing of a primary transcript which may have a length of >100 nucleotides.

Suitably, the isolated plant viral miRNAs of the invention comprise a nucleotide sequence that is capable of modulating the expression and/or activity of one or more plant defence nucleic acids.

Typically, said isolated plant viral RNA molecule comprises a nucleotide sequence that is capable of at least partially reducing, mitigating, silencing, suppressing, inhibiting, or otherwise decreasing the expression and/or activity of one or more plant defence nucleic acids.

By “plant defence nucleic acid” is intended a plant nucleic acid that encodes a plant protein that confers reduced susceptibility to plant pathogens (e.g., viruses). Exemplary plant defence nucleic acids that encode proteins conferring reduced susceptibility to plant pathogens include, but are not limited to, nucleic acids or mutated versions or orthologs of eukaryotic initiation factor 4 E (eIF4E; e.g., CUM2), N immune receptor, a chloroplastic protein interacting with the N immune receptor (NRIP1), resistance protein to tomato mosaic virus (Tm-1, Tm-2, Tm-22), resistance protein to potato virus X (Rx), rice yellow mottle virus resistance proteins (RYMV1, RYMV2), wheat streak mosaic_virus resistance protein (Wsm1), barley yellow dwarf virus resistance protein (Ryd4), NAC domain transmembrane proteins required for tobamovirus (TOM1, TOM2A, TOM3), systemic movement protein required for tobamovirus (VSM1), lectin-like protein and heat shock protein for potyvirus (RTM1, RTM2), Pathogen-related protein 5 (PR5), Lectin protein kinase (Lec), Lesion inducing protein (hypersensitive response inducing), Vanguard 1 (VGD1), Tombusvirus replication protein 1 (Tom1), NRPD1B, Expansin8 (EXP8), Brassinosteroid signalling regulator (BEH1), and Brassinosteroid signalling regulator (ATBS1), or orthologs of these (see also, Truniger and Aranda, Recessive resistance to plant viruses. Adv. Virus Res. 75:119-59, 2009), and other genes involved in hypersensitive response (HR)/programmed cell death and/or other plant defence genes acting against biotrophic pathogens.

In one embodiment, said plant defence nucleic acid is a plant viral defence nucleic acid. Suitably, said plant viral defence nucleic acid is HVA22d. It will be appreciated that HVA22d refers to an abscisic acid-inducible gene that encodes an abscisic acid (ABA)-responsive protein.

As used herein, the terms “silencing”, “inhibiting” or “suppressing” are used interchangeably to denote the down-regulation of the expression and/or activity of the plant defence nucleic acid relative to its expression and/or activity in a corresponding plant or plant cell that does not comprise the plant viral miRNA.

Typically, the plant viral miRNA does not encode a functional peptide or a protein encoded by a genome, but maybe located within a coding region of a plant viral genome. Accordingly, the miRNA comprises a nucleotide sequence that is referred to herein as “non-translated”.

Suitably, the plant viral miRNAs require a dicer and/or one or more dicer-like (DCL) proteins for their processing and/or production. It will be appreciated that the plant viral miRNAs typically use their plant host machinery for their processing and/or production. Typically, although not exclusively, the processing and/or production of the mature plant viral miRNA is mediated by DCL-1, DCL-2, DCL-4, and/or Argonaute protein-1 (AGO1). Suitably, DCL-1 processes viral RNA to produce the miRNA precursor in the nucleus of a plant cell. DCL-2 and/or DCL-4 typically process the miRNA precursor to produce the mature miRNA. Once processed, the mature miRNA is typically present in the cytoplasm. Thus, it will be appreciated that the processing by DCL-2 and/or DCL-4 may occur in the cytoplasm. Alternatively, the processing by DCL-2 and/or DCL-4 may occur in the nucleus after which the mature plant viral miRNA is transported from the nucleus to the cytoplasm.

In one preferred form, said isolated RNA molecule is encoded by the genome of a plant RNA virus, for example, a positive sense single-stranded (ss) RNA virus (ssRNA+), a negative sense single-stranded RNA virus (ssRNA−) or a double-stranded RNA virus (dsRNA). Suitably, said isolated RNA molecule is encoded by the genome of a virus of the Potyviridae, Virgaviridae, Bunyaviridae, or Reoviridae families. Accordingly, said isolated RNA molecule may be encoded by the genome of a virus of the genus Potyvirus, Ipomovirus, Macluravirus, Rymovirus, Tritimovirus, Bymovirus, Tobamovirus, Tospovirus, or Fijivirus. Suitably, said isolated RNA molecule is encoded by the genome of a virus of a species of Turnip mosaic virus (TuMV), Tobacco mosaic virus (TMV), Tomato spotted wilt virus (ToSWV), or Fiji disease virus.

Non-limiting examples of the isolated plant viral RNA molecules of the invention are set forth in SEQ ID NOs: 1-82 and their complements SEQ ID NOS: 139-220, respectively (Table 1).

It will be appreciated that said plant viral miRNA molecule may be chemically-synthesised de novo, rather than transcribed from a DNA sequence.

Chemical synthesis of RNA is well known in the art. Non-limiting examples include RNA synthesis using TOM amidite chemistry, 2-cyanoethoxymethyl (CEM), a 2′-hydroxyl protecting groups and fast oligonucleotide deprotecting groups.

It will also be appreciated that the invention contemplates nucleic acid molecules (e.g., RNA or DNA) complementary to or at least partly complementary to the plant viral miRNAs of the invention. Complementary or at least partly complementary nucleic acid molecules may be in DNA or RNA form.

By “at least partly complementary” is meant having at least 60%, at least 70%, at least 75%, at least 80%, at least 90%, or at least 91%, 92%, 93%, 94%, 95%, 96%, 97%, 98%, or at least 99% sequence identity with a nucleotide sequence of a plant viral miRNA molecule.

The invention also provides a modified plant viral miRNA. A modified plant viral miRNA may be altered by, complexed, labelled or otherwise covalently or non-covalently coupled to one or more other chemical entities. In some embodiments, the chemical entity may be bonded, linked or otherwise attached directly to the plant viral miRNA, or it may be bonded, linked or otherwise attached to the plant viral miRNA via a linking group (e.g., a spacer).

Examples of such chemical entities include, but are not limited to, incorporation of modified bases (e.g., inosine, methylinosine, pseudouridine and morpholino), sugars and other carbohydrates such as 2′-O-methyl and locked nucleic acids (LNA), amino groups and peptides (e.g., peptide nucleic acids (PNA)), biotin, cholesterol, fluorophores (e.g., FITC, Fluoroscein, Rhodamine, HEX, FAM, TET, and Oregon Green) radionuclides and metals, although without limitation thereto (Fabani and Gait, 2008; You et al., 2006; Summerton and Weller, 1997). A more complete list of possible chemical modifications can be found at http://www.oligos.com/ModificationsList.htm.

In one particular embodiment, the modified plant viral miRNA is an “antisense inhibitor”. By “antisense inhibitor” is meant a nucleic acid sequence that is either complementary to or at least partly complementary to the plant viral miRNA molecule. The antisense inhibitor pairs with the plant viral miRNA and interferes with interactions such as, but not limited to, plant viral miRNA-mRNA and plant viral miRNA-RNA interactions.

In another particular embodiment, the modified plant viral miRNA is a “point mutant”. By “point mutant” is meant a plant viral miRNA where 1 or 2 nucleotides have been removed, substituted or otherwise altered. Point mutants of plant viral miRNAs or their targets can be employed to study the function of plant viral miRNAs in plant disease or to decrease the affinity of plant viral miRNAs to their targets (e.g., plant defence nucleic acids). Small RNA molecules involved in plant disease processes, including plant viral miRNAs, may have “seed-sequences”. By “seed-sequences” is meant nucleic acid sequences that comprise 2-7 nucleotides and are involved in target recognition. Increasing the mismatch in these sequences is predicted to significantly decrease the gene regulation function of plant viral miRNAs.

In yet another particular embodiment, the modified plant viral miRNA molecule is a “plant viral miRNA sponge”. By “plant viral miRNA sponge” is meant a genetically encoded competitive plant viral miRNA inhibitor that may be stably expressed in a cell, such as a plant cell. The plant viral miRNA sponge binds to the plant viral miRNA thereby preventing it from binding its mRNA target in a technique called “sponging”. Plant viral miRNA sponges may be produced using methods such as the ones described in Cohen, 2009, Ebert et al., 2007, Hammond, 2007 and Rooij et al., 2008. It will be appreciated that a plant viral miRNA sponge may bind to, soak up and/or inhibit a specific plant viral miRNA and/or a family of plant viral miRNAs.

In still yet another particular embodiment, the modified plant viral miRNA is a “plant viral miRNA mimic”. A “plant viral miRNA mimic” is a single-stranded RNA oligonucleotide that is complementary to, or at least partly complementary to, the plant viral miRNA. The plant viral miRNA mimic may inactivate viral plant viral mRNAs through complementary base-pairing. Plant viral miRNA mimics may be particularly suitable for studying the effects of certain plant viral miRNAs in a plant host.

The invention also provides a fragment of a plant viral miRNA of the invention. By “fragment” is meant a portion, domain, region or sub-sequence of a plant viral miRNA which comprises one or more structural and/or functional characteristics of a plant viral miRNA molecule. By way of example only, a fragment may comprise at least 5, at least 6, at least 7, at least 8, at least 9, at least 10, at least 12, at least 14, at least 16, at least 18, at least 19, at least 20, at least 21, at least 22, or at least 23 nucleotides of a plant viral miRNA.

It will be appreciated that the plant viral miRNAs can be chemically modified to facilitate penetration into a cell. Examples of such modifications include, but are not limited to, conjugation to cholesterol, Morpholino, 2′ O-methyl, PNA or LNA.

Modified plant viral miRNAs also include “variants” of the plant viral miRNAs of the invention. Variants include RNA or DNA molecules comprising a nucleotide sequence at least 70%, 75%, 80%, 85%, 90%, 95%, 96%, 97%, 98%, or 99% identical to a nucleotide sequence of a plant viral miRNA such as described in Table 1 (SEQ ID NOs: 1-82 and their complements SEQ ID NOS: 139-220, respectively). Such variants may include one or more point mutations, nucleotide substitutions, deletions or additions.

In further aspects, the invention provides methods of producing the isolated RNA molecule, said method including the step of isolating one or more of said isolated RNA molecules from a nucleic acid sample obtained from a plant pathogen or a plant infected with said plant pathogen.

It will be appreciated that plant viral miRNA molecules appear to be a hitherto unknown form of small, single stranded viral RNA molecules that are encoded by plant viruses. Accordingly, plant viral miRNA molecules may be isolated, identified, purified or otherwise obtained from a number of different plant viruses, such as DNA viruses and RNA viruses. Non-limiting examples of plant viruses may, for example, be found at http://www.dpvweb.net/dpv/dpvtaxonidx.php. Preferably, the virus is an RNA virus (e.g., a double-stranded or single-stranded RNA virus).

Broadly, such methods may include analysis of nucleic acid samples obtained from a plant and/or a plant virus, and/or bioinformatic analysis of genome sequence information.

Nucleic acid-based detection may utilise one or more techniques including nucleic acid sequence amplification, probe hybridisation, mass spectrometry, nucleic acid arrays and nucleotide sequencing, although without limitation thereto.

In one embodiment, the invention contemplates nucleic acid sequence amplification and subsequent detection of one or more amplification products.

Nucleic acid amplification techniques are well known to the skilled addressee, and include polymerase chain reaction (PCR) and ligase chain reaction (LCR) as for example described in Chapter 15 of Ausubel et al. CURRENT PROTOCOLS IN MOLECULAR BIOLOGY (John Wiley & Sons NY, 1995-1999); strand displacement amplification (SDA) as for example described in U.S. Pat. No. 5,422,252; rolling circle replication (RCR) as for example described in Liu et al., 1996, J. Am. Chem. Soc. 118 1587 and International application WO 92/01813 and by Lizardi et al., in International Application WO 97/19193; nucleic acid sequence-based amplification (NASBA) as for example described by Sooknanan et al., 1994, Biotechniques 17 1077; Q-β replicase amplification as for example described by Tyagi et al., 1996, Proc. Natl. Acad. Sci. USA 93 5395 and helicase-dependent amplification as described in International Publication WO2004/02025.

The abovementioned are examples of nucleic acid sequence amplification techniques but are not presented as an exhaustive list of techniques. Persons skilled in the art will be well aware of a variety of other applicable techniques as well as variations and modifications to the techniques described herein.

For example, the invention contemplates use of particular techniques that facilitate quantification of nucleic acid sequence amplification products such as by “Competitive PCR”, or techniques such as quantitative Real-Time PCR and reverse transcriptase PCR (“qPCR” and “qRT-PCR”, respectively) amplification.

As used herein, an “amplification product” is a nucleic acid generated by a nucleic acid sequence amplification technique as hereinbefore described.

Detection of amplification products may be achieved by detection of a probe hybridised to an amplification product, by direct visualisation of amplification products by way of agarose gel electrophoresis, nucleotide sequencing of amplification products or by detection of fluorescently-labelled amplification products.

As used herein, a “probe” is a single- or double-stranded oligonucleotide or polynucleotide, one and/or the other strand of which is capable of hybridising to another nucleic acid, to thereby form a “hybrid” nucleic acid.

Probes and/or primers of the invention may be labelled, for example, with biotin or digoxigenin, with fluorochromes or donor fluorophores such as FITC, TRITC, Texas Red, TET, FAM6, HEX, ROX or Oregon Green, acceptor fluorophores such as LC-Red640, enzymes such as horseradish peroxidase (HRP) or alkaline phosphatase (AP) or with radionuclides such as ¹²⁵I, ³²P, ³³P or ³⁵S to assist detection of amplification products by techniques as are well known in the art.

As used herein, “hybridisation”, “hybridise” and “hybridising” refers to formation of a hybrid nucleic acid through base-pairing between complementary or at least partially complementary nucleotide sequences under defined conditions, as is well known in the art. Normal base-pairing occurs through formation of hydrogen bonds between complementary A and T or U bases, and between G and C bases. It will also be appreciated that base-pairing may occur between various derivatives of purines (G and A) and pyrimidines (C, T and U). Purine derivatives include inosine, methylinosine and methyladenosines. Pyrimidine derivatives include sulfur-containing pyrimidines such as thiouridine and methylated pyrimidines such as methylcytosine. For a detailed discussion of the factors that generally affect nucleic acid hybridisation, the skilled addressee is directed to Chapter 2 of CURRENT PROTOCOLS IN MOLECULAR BIOLOGY, supra.

More specifically, the terms “anneal” and “annealing” are used in the context of primer hybridisation to a nucleic acid template for a subsequent primer extension reaction, such as occurs during nucleic acid sequence amplification or nucleotide sequencing, as for example described in Chapter 15 of CURRENT PROTOCOLS IN MOLECULAR BIOLOGY, supra.

In another embodiment, detection may be performed by melting curve analysis using probes incorporating fluorescent labels that hybridise to amplification products in a sequence amplification reaction. A particular example is the use of Fluorescent Resonance Energy Transfer (FRET) probes to hybridise with amplification products in “real time” as amplification products are produced with each cycle of amplification.

In yet another embodiment, the invention contemplates use of melting curve analysis whereby nucleic acid-intercalating dyes such as Ethidium Bromide (EtBr) or SYBR Green I bind amplification products and fluorescence emission by the intercalated complexes is detected.

Particularly for the purpose of detection, although without limitation thereto, the present invention provides a kit comprising one or more probes and/or primers that facilitate detection of (i) a plant viral miRNA, or a fragment thereof; (ii) a precursor of the plant viral miRNA, or a fragment thereof; and/or (iii) a plant defence nucleic acid that is modulated by the plant viral miRNA, or a fragment thereof. Said kit may further comprise other reagents such as a thermostable DNA polymerase, positive and/or negative nucleic acid control samples, molecular weight markers, detection reagents such as for colorimetric detection or fluorescence detection of amplification products and/or reaction vessels such as microtiter plates.

According to another aspect, there is provided a genetic construct comprising or encoding one or a plurality of the same or different plant viral miRNAs, miRNA precursors, modified plant viral miRNAs, at least partly complementary DNA or RNA molecules, or fragments thereof.

It will be appreciated that said plant viral miRNA molecules may be oriented in tandem repeats or with multiple copies of each plant viral miRNA sequence.

As used herein, a “genetic construct” is any artificially constructed nucleic acid molecule comprising heterologous nucleotide sequences.

A genetic construct is typically in DNA form, such as a phage, plasmid, cosmid, artificial chromosome (e.g., a YAC or BAC), although without limitation thereto. The genetic construct suitably comprises one or more additional nucleotide sequences, such as for assisting propagation and/or selection of bacterial or other cells transformed or transfected with the genetic construct.

In one particular embodiment, the genetic construct is a DNA expression construct that comprises one or more regulatory sequences that facilitate transcription of one or more plant viral miRNA molecules, modified plant viral miRNA molecules or fragments thereof.

Such regulatory sequences may include promoters, enhancers, polyadenylation sequences, splice donor/acceptor sites, although without limitation thereto.

Suitable promoters may be selected according to the cell or organism in which the plant viral miRNA molecule is to be expressed. Promoters may be selected to facilitate constitutive, conditional, tissue-specific, inducible or repressible expression as is well understood in the art. Examples of constitutive promoters are the Cauliflower mosaic virus (CaMV) 35S promoter, the CaMV 19S promoter, the plant ubiquitin 1 promoter, the Smas promoter, the rubisco promoter and other transcription initiation regions from various plant genes known to those of skill in the art.

Examples of inducible promoters include the Adh1 promoter, the Hsp promoter and the PPDK promoter. Promoters may also initiate transcription in certain tissues, such as leaves, roots, fruits, seeds or flowers. Specific examples of promoters including tissue-preferred, leaf-preferred and root-preferred promoters may be found in published US Patent Application 20060130176.

The present invention also provides a host cell comprising the aforementioned nucleic acid construct.

By “host cell” is meant a cell which contains an introduced nucleic acid construct and supports the replication and/or expression of the construct. Host cells may be prokaryotic cells such as E. coli, or eukaryotic cells such as fungi, yeast, insect, or mammalian cells. Alternatively, the host cells are plant cells, including (but not limited to) monocotyledonous or dicotyledonous plant cells. An example of a monocotyledonous plant cell is a maize cell, while tomato and peanut cells are examples of dicotyledonous plant cells.

In another aspect, the invention provides a method of identifying a plant defence nucleic acid, said method including the step of identifying a plant defence nucleic acid that is modulated by one or more of the isolated plant viral miRNAs of the invention.

Suitably, the plant viral miRNA has modulated the expression and/or activity of the plant defence nucleic acid. Preferably, the plant viral miRNA has at least partially reduced, lowered, or decreased the expression and/or activity of the plant defence nucleic acid.

The invention also provides a method of modifying a plant defence nucleic acid, said method including the step of modifying a nucleotide sequence of the plant defence nucleic acid to be at least partially resistant to modulation by the plant viral miRNA.

A number of different methods may be employed to modify, alter, or otherwise change the plant defence nucleic acid and it is recognised that methods of the present invention do not depend on the incorporation of an entire polynucleotide into the genome, only that the plant and/or plant cell is altered as a result of the introduction of the polynucleotide into a cell. Alterations to the genome of the present invention include, but are not limited to, additions, deletions, and substitutions of nucleotides into the genome. While the methods of the present invention do not depend on additions, deletions, and substitutions of any particular number of nucleotides, it is recognised that such additions, deletions, or substitutions comprise at least one nucleotide.

Suitably, said plant defence nucleic acid is modified by introducing a silent mutation into a region that is recognised by the isolated plant viral mRNA. By “silent mutation” is meant that the mutation alters the nucleotide sequence of the nucleic acid without altering the polypeptide sequence of the corresponding protein.

In one particular embodiment, said plant defence nucleic acid is modified by zinc finger gene targeting (see for example Osakabe et al., 2010 and Zhang et al., 2010). Zinc finger gene targeting uses zinc finger nucleases (ZFNs), a class of engineered DNA-binding proteins, to facilitate targeted modifications of the genome by creating double-strand breaks in the genome at specific locations. A skilled person will appreciate that zinc finger gene targeting may, for example, be used to generate cell lines comprising targeted gene deletions, integrations, and/or mutations (e.g., a silent point mutation in HVA22d). Further information may be found at http://www.sigmaaldrich.com/life-science/zinc-finger-nuclease-technology.html.

The invention also provides an isolated modified plant defence nucleic acid that has been modified as hereinbefore described.

In another aspect, the invention provides a method of reducing a susceptibility of a plant to a pathogen, said method including the step of introducing an isolated modified plant defence nucleic acid into the plant to thereby reduce, decrease, or mitigate the susceptibility of said plant to said pathogen.

In a further aspect, the invention provides a method of reducing a susceptibility of a plant to a pathogen, said method including the step of introducing a decoy target sequence into the plant to thereby reduce, decrease, or mitigate the susceptibility of the plant to the pathogen, wherein the decoy target sequence binds, anneals to, hybridises to, or otherwise recognises and captures one or more of the isolated plant viral miRNAs of the invention.

Thus, in some embodiments, the methods of the invention involve introducing a nucleic acid into a plant in such a manner that the nucleic acid gains access to the interior of at least one cell of the plant. Methods for introducing nucleic acids into plants are known in the art including, but not limited to, stable transformation methods, transient transformation methods, and virus-mediated methods.

“Stable transformation” is intended to mean that the nucleic acid construct introduced into a plant integrates into the genome of the plant and is capable of being inherited by the progeny thereof. “Transient transformation” is intended to mean that the nucleic acid is introduced into the plant and does not integrate into the genome of the plant.

Transformation protocols as well as protocols for introducing the nucleic acid into plants may vary depending on the type of plant or plant cell targeted for transformation. In some embodiments, the methods of the present invention involve transformation protocols suitable for introducing nucleic acids into monocots.

Suitable transformation methods of introducing nucleic acids into plant cells include microinjection (Crossway et al. (1986) Biotechniques 4320-334), electroporation (Riggs et al. (1986) Proc. Natl. Acad. Sci. USA 83:5602-5606), Agrobacterium-mediated transformation (U.S. Pat. Nos. 5,563,055 and 5,981,840), direct gene transfer (Paszkowski et al. (1984) EMBO J. 3:2717-2722), ballistic particle acceleration (see, e.g., U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Led 1 transformation (WO 00/28058).

Methods are also known in the art for the targeted insertion of a nucleic acid at a specific location in the plant genome. In one embodiment, the insertion of the nucleic acid at a desired genomic location is achieved using a site-specific recombination system. See, for example, WO 99/25821, WO 99/25854, WO 99/25840, WO 99/25855, and WO 99/25853, all of which are herein incorporated by reference. Briefly, a nucleic acid can be contained in a transfer cassette flanked by two non-recombinogenic recombination sites. The transfer cassette is introduced into a plant having stably incorporated into its genome a target site that is flanked by two non-recombinogenic recombination sites that correspond to the sites of the transfer cassette. An appropriate recombinase is provided and the transfer cassette is integrated at the target site. The nucleic acid of interest is thereby integrated at a specific chromosomal position in the plant genome.

In another aspect, the invention provides a method of reducing a susceptibility of a plant population to a pathogen, said method including the step of selecting for at least one plant that comprises a naturally occurring plant defence nucleic acid that is not susceptible to modulation by the plant viral miRNA, which thereby has a reduced, decreased, or mitigated susceptibility to said pathogen, and using the at least one plant in plant breeding.

It will be appreciated that the pathogen may be selected from the group consisting of a virus, a fungus, an oomycete, or a bacterium. Suitably the pathogen is a virus, such as an RNA virus.

By “breeding a plant”, “plant breeding” or “conventional plant breeding” is meant the creation of a new plant variety or cultivar by hybridisation of two donor plants, at least one of which carries a trait of interest, followed by screening and field selection. Such methods are not reliant upon transformation with recombinant DNA in order to express a desired trait. However, it will be appreciated that in some embodiments, the donor plant may carry the trait of interest as a result of transformation with recombinant DNA which imparts the trait.

It will be appreciated by a person of skill in the art that a method of plant breeding typically comprises identifying a parent plant which comprises at least one genetic element associated with or linked to a desired trait (e.g., a silent mutation in the HVA22d nucleic acid). This may include initially determining the genetic variability in the genetic element between different plants to determine which alleles or polymorphisms would be selected for in the plant breeding method of the invention. This may also be facilitated by use of additional genetic markers associated with the desired trait that are useful in marker-assisted breeding methods.

By way of example only, a plant breeding method may include the following steps:

(a) identifying a first parent plant and a second parent plant, wherein at least one of the first and second parent plants comprise at least one genetic element associated with or linked to a desired trait (e.g., a silent mutation in the HVA22d nucleic acid);

(b) pollinating the first parent plant with pollen from the second parent plant, or pollinating the second parent plant with pollen from the first parent plant;

(c) culturing the plant pollinated in step (b) under conditions to produce progeny plants; and

(d) selecting progeny plants that possess the desired trait.

It will be appreciated that plants comprising a genetic element that is associated with, or linked to, a desired trait (e.g., a silent mutation in HVA22d) may be screened for using sequential PCR and/or single nucleotide polymorphism (SNP) detection.

It will be also be appreciated by those skilled in the art that once progeny plants have been obtained (e.g., F1 hybrids), which may be heterozygous or homozygous, these heterozygous or homozygous plants may be used in further plant breeding (e.g., backcrossing with plants of parental type or further inbreeding of F1 hybrids).

In particular embodiments, the present invention may be used in combination with other genetic approaches to confer improved disease resistance. Examples of such genetic approaches include, but are not limited to, (i) silencing, down-regulating or otherwise suppressing the expression and/or activity of a negative regulator of plant defence signalling; (ii) increasing, inducing, upregulating or otherwise enhancing the expression and/or activity of a positive defence signalling regulator, and/or (iii) inducing, upregulating, or otherwise enhancing the expression and/or activity of a defence gene that confers viral resistance.

In certain embodiments that relate to bioinformatic analyses of genome sequence information, the invention provides a computer-readable storage medium or device encoded with structural and functional information of one or more plant viral miRNAs.

The structural and functional information may be host plant virus, nucleotide sequence of the precursor and/or the mature plant viral miRNA, sequence length, target nucleic acid(s) and plant viral miRNA recognition sequence, although without limitation thereto.

A computer-readable storage medium may have computer readable program code components stored thereon for programming a computer (e.g., any device comprising a processor) to perform a method as described herein. Examples of such computer-readable storage media include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one having ordinary skill in the art, notwithstanding possibly significant effort and many design choices motivated by, for example, available time, current technology, and economic considerations, when guided by the concepts and principles disclosed herein will be readily capable of implementing the invention by generating necessary software instructions, programs and/or integrated circuits (ICs) with minimal experimentation.

Typically, the computer-readable storage medium or device is part of a computer or computer network capable of interrogating, searching or querying a genome sequence database.

In one example, a bioinformatic method may utilise a high performance computing station which houses a local mirror of the UCSC Genome Browser.

The invention also provides a nucleic acid array comprising a plurality of the isolated RNA molecules, immobilised, affixed or otherwise mounted to a substrate.

By “nucleic acid array” is a meant a plurality of nucleic acids, preferably ranging in size from 10, 15, 20 or 50 bp to 250, 500, 700, or 900 kb, immobilised, affixed or otherwise mounted to a substrate or solid support. Typically, each of the plurality of nucleic acids has been placed at a defined location, either by spotting or direct synthesis. In array analysis, a nucleic acid-containing sample is labelled and allowed to hybridise with the plurality of nucleic acids on the array. Nucleic acids attached to arrays are referred to as “targets” whereas the labelled nucleic acids comprising the sample are called “probes”. Based on the amount of probe hybridised to each target spot, information is gained about the specific nucleic acid composition of the sample. The major advantage of gene arrays is that they can provide information on thousands of targets in a single experiment and are most often used to monitor gene expression levels and “differential expression”.

“Differential expression” indicates whether the level of a particular plant viral miRNA in a sample is higher or lower than the level of that particular plant viral miRNA in a normal or reference sample.

The physical area occupied by each sample on a nucleic acid array is usually 50-200 μm in diameter thus nucleic acid samples representing entire genomes, ranging from 3,000-32,000 genes, may be packaged onto one solid support. Depending on the type of array, the arrayed nucleic acids may be composed of oligonucleotides, PCR products or cDNA vectors or purified inserts. The sequences may represent entire genomes and may include both known and unknown sequences or may be collections of known miRNA sequences. Using array analysis, the expression profiles of uninfected and virally infected plants, treated and untreated cell cultures, and developmental stages of a plant can be compared.

In one embodiment, gene profiling, such as but not limited to using a plant viral miRNA array, is used to identify mRNAs whose expression and/or activity shows a positive or inverse correlation with the expression of a specific plant viral miRNA.

It will be appreciated that an absence of plant viral miRNA expression could correlate with a presence of mRNA expression, or vice versa. Alternatively, a presence of plant viral miRNA expression could correlate with a presence of mRNA expression or an absence of plant viral miRNA expression could correlate with an absence of mRNA expression. Furthermore, a level of plant viral miRNA expression could correlate with a level of mRNA expression, whether directly or inversely. It will be appreciated that a level of expression may be measured as a quantitative or a relative expression level.

One further aspect of the invention provides antibodies which bind, recognise and/or have been raised against a plant viral miRNA of the invention, inclusive of fragments and modified plant viral miRNA molecules.

Antibodies may be monoclonal or polyclonal. Antibodies also include antibody fragments such as Fc fragments, Fab and Fab′ 2 fragments, diabodies and

ScFv fragments. Antibodies may be made in a suitable production animal such as a mouse, rat, rabbit, sheep, chicken or goat.

The invention also contemplates recombinant methods of producing antibodies and antibody fragments. For example, antibodies to RNA molecules have been produced by a method utilising a synthetic phage display library approach to select RNA-binding antibody fragments (Ye et al., 2008).

As is well understood in the art, antibodies may be conjugated with labels selected from a group including an enzyme, a fluorophore, a chemiluminescent molecule, biotin, radioisotope or other label.

Examples of suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution or with a suitable chromogenic or chemiluminescent substrate.

Examples of chromogens include diaminobanzidine (DAB), permanent red, 3-ethylbenzthiazoline sulfonic acid (ABTS), 5-bromo-4-chloro-3-indolyl phosphate (BCIP), nitro blue tetrazolium (NBT), 3,3′,5,5′-tetramethyl benzidine (TNB) and 4-chloro-1-naphthol (4-CN), although without limitation thereto.

A non-limiting example of a chemiluminescent substrate is Luminol™, which is oxidised in the presence of horseradish peroxidase and hydrogen peroxide to form an excited state product (3-aminophthalate).

Fluorophores may be fluorescein isothiocyanate (FITC), tetramethylrhodamine isothiocyanate (TRITC), allophycocyanin (APC), Texas Red (TR), Cy5 or R-Phycoerythrin (RPE), although without limitation thereto.

Radioisotope labels may include ¹²⁵I, ¹³¹I, ⁵¹Cr and ⁹⁹Tc, although without limitation thereto.

Other antibody labels that may be useful include colloidal gold particles and digoxigenin.

This aspect also provides a kit comprising one or more of the isolated RNA molecules, an antibody, and one or more detection reagents.

### EXAMPLES

### Example 1: Repression of Plant Defence Through Viral miRNA

### Materials and Methods

### Plant Material

The Salk mutants were obtained from The Salk Institute Genome Analysis Laboratory (La Jolla, Calif.).

Cloning of microRNAs

Total RNA was isolated using Trizol Reagent (MRC Inc.). Small RNA was fractionated using PureLink™ miRNA isolation Kit (Invitrogen). Small RNA was run on 15% PAGE and the 10-40 nt size band was cut. RNA was eluted by gently agitating the chopped gel overnight in ˜300 μl of water at 4° C. The RNA-containing supernatant was separated by centrifugation for 5 min and 2.5 volumes of 2-butanol were added and centrifuged. Lower phase was isolated and proceeded for Chloroform extraction and ethanol precipitation with Glycogen as carrier. The small RNAs were polyadenylated, using NCode™ miRNA First-Strand cDNA Synthesis and qRT-PCR Kit (Invitrogen). MicroRNAs were PCR amplified using 3′ oligo-dT primers and 5′ microRNA specific 14 nt primer. Amplified product was cloned in TA cloning vector (Invitrogen) and screened through sequencing.

### Isolation of Nuclei and Nuclear RNA Extraction

Nuclei were isolated using modified version of a published protocol (Tomasz & Meier, 2006). Arabidopsis leaf tissue (1 g) was ground finely in liquid nitrogen and 5 ml of Nuclei Isolation Buffer (NIB) (20 mM KCl, 20 mM HEPES, 0.6% TritonX-100 and 30 mM β-Mercaptoethanol) was added. The homogenised mixture was kept on ice for 10-15 min. The cellular debris was removed by sieving through 4-5 layers of Kim wipes dust-free tissues (KIMTECH*, Kimberley-Clark). The flow through was centrifuged at 1000×g for 10 min at 4° C. The supernatant was discarded, pellet containing nuclei was dissolved in 400 μl of NIB, placed on top of 800 μl of 1.5 M sucrose cushion prepared in NIB and centrifuged at 12, 000×g for 10 min at 4° C. The pellet containing semi-pure nuclei was washed 2-3 times with NIB and Trizol Reagent (MRC Inc.) was added to the pellet for RNA extraction.

### Northern Blot Hybridisation

Small RNA Northern blot hybridisation was carried out using 15% denaturing polyacrylamide gel. Total RNA was isolated from the samples using Trizol Reagent (MRC inc.) and the small RNA fraction was separated using Purelink™ miRNA isolation kit from Invitrogen. Small RNA fraction (5-15 μg) was run on gel and transferred to nylon membrane using a semi-dry electro-blotter (Bio-Rad). Probes were labelled with [α-³²P] CTP using end labelling method. Hybridisation was carried out at 50° C.

For high molecular weight RNA Northern, total RNA was isolated using SV Total RNA isolation system (Promega, Madison, Wis.). Total RNA (10 μg) was run on 1% agarose gel containing 2.2 M formaldehyde and transferred onto nylon membrane through capillary-based transfer in 10×SSC. Hybridisation was carried out at 65° C.

qRT-PCR Protocol for Detecting Gene Expression

Plant samples (˜30 plants), in three biological replicates (˜10 plants each) for each treatment were collected. Total RNA was extracted using SV RNA isolation kit (Promega, Madison, Wis.). cDNA was synthesised from 1.5 μg of RNA using Superscript RT III reverse transcriptase kit (Invitrogen, Carlsbad, Calif.). Real-time PCR was carried out using SYBR-green master PCR mix (Perkin-Elmer Applied Biosystems) in an ABI model 7900 sequence detection system (Perkin-Elmer Applied Biosystems, Foster City, Calif.). The control qRT-PCR primers used were, β-Actin 7-Reverse (At5g09810) 5′GAGGAAGAGCATTCCCCTCGTA′3 (SEQ ID NO:83) and β-Actin 2-Reverse (At3g18780) 5′GATGGCATGGAGGAAGAGAGAAAC′3 (SEQ ID NO:84) with a universal Actin forward primer 5′AGTGGTCGTACAACCGGTATTGT′3 (SEQ ID NO:85). RT-Q-PCR results were analyzed using the sequence detection software SDS version 2.2 (Perkin-Elmer Applied Biosystems). GFP Forward primer was 5′ AACCATTACCTGTCCACACAATCTG ‘3 (SEQ ID NO:86) and GFP reverse primer was 5’ ATAGTTCATCCATGCCATGTGTAATC ′3 (SEQ ID NO:87).

### Microarray Detection of Small RNAs

The microRNA microarray service was commercially provided by LC Sciences, LLC Houston, Tex. RNA was extracted from two biological replicates (10 plants each) for each Turnip mosaic virus infected and uninfected (Col-0) WT plants (Gene Expression Omnibus accession number GSE22583). RNA from both biological replicates for each treatment was probed for detection of predicted microRNA sequences.

### Results

### Localisation of Viral RNA in the Nucleus

MicroRNA processing occurs in the nucleus (Park et al., 2005), and there is some evidence that potyviral components translocate to the nucleus during the virus infection cycle. Green fluorescent protein (GFP) fusions of NIa and NIb (viral RdRps), which act as major enzymes in potyviral replication, have revealed that these proteins remain localised in the host nuclei during the course of virus infection (Restrepo et al., 1990). The Nib sequence of Tobacco etch potyvirus (TEV) contains two autonomous nuclear localisation signal (NLS) sequences, NLS I and NLS each of which is important for a successful viral infection. Mutation in any one NLS sequence results in loss of viral infectivity (Li et al., 1997).

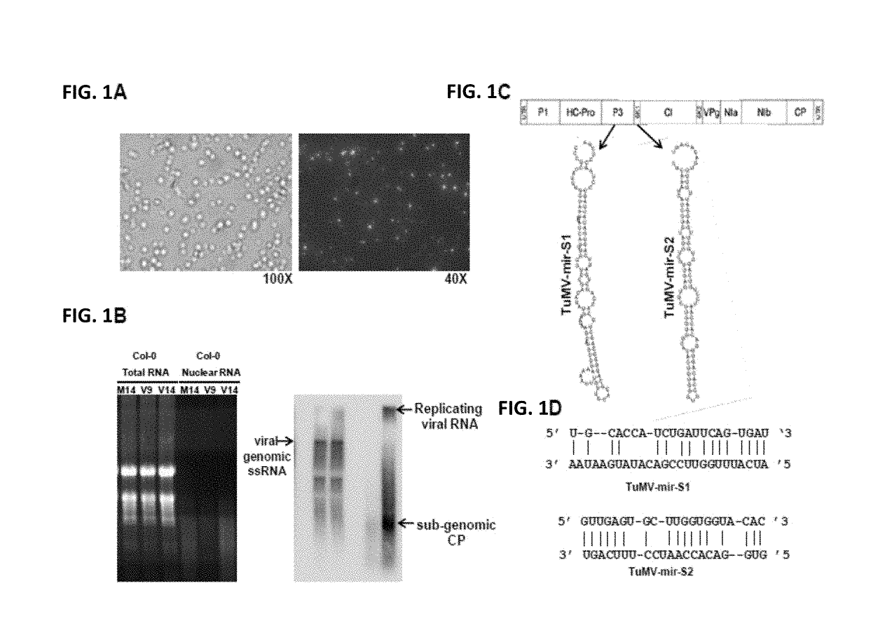

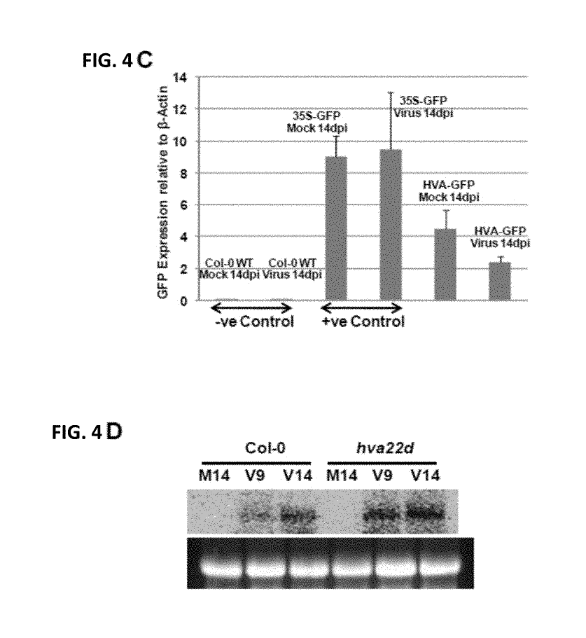

The first question we therefore addressed was whether our TuMV isolate could be detected in the nucleus of Arabidopsis cells. We isolated intact nuclei (FIG. 1A) from TuMV-infected and uninfected Arabidopsis plants, and purified nuclear RNA fractions. Hybridisation of a nuclear RNA blot with a DNA probe encoding viral coat protein surprisingly showed major localisation of the double-stranded replicative form of TuMV in the nucleus (FIG. 1B). Northern blot analysis also indicated the presence of sub-genomic TuMV coat protein transcript in the nucleus of virus-infected Arabidopsis cells (FIG. 1B). Presence of replicative double-stranded viral RNA in the nucleus was confirmed by using strand-specific oligo probe for plus strand detection and strand specific RT-PCR to confirm the presence of negative strand in nuclear RNA extract (FIG. 6), and is consistent with the previous observation that viral replication-associated proteins NIa and NIb reside in the nucleus during the course of virus infection. As expected, the level of single-stranded, positive strand viral RNA is below the detection threshold in the nuclear RNA fraction (FIG. 1B), suggesting that this viral form is rapidly translocated to the cytoplasm.

### Prediction and Detection of TuMV-mir-S1 and TuMV-mir-S2

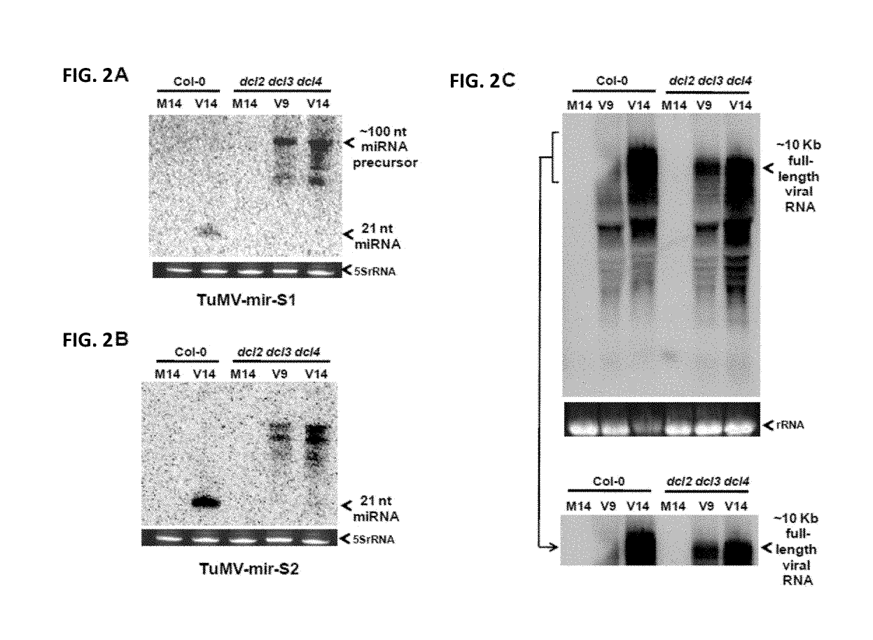

The TuMV-BRS1 isolate we used in this study has not been characterised. The viral genome was sequenced (HM544042) using degenerate primers and subjected to microRNA precursor prediction with ProMiR II and Mfold (Jin-Wu et al., 2006; Zuker, 2003). A total of 13 microRNA precursors were predicted on the plus strand and 18 on the minus strand of the virus. To start with, these precursor sequences were aligned against preexisting microRNA sequences in the miRBASE (Griffiths-Jones et al., 2008; Griffiths-Jones et al., 2006) microRNA database to see resemblance with any pre-existing microRNAs. Using this search we selected a number of mature microRNA sequences with some structural similarity to the existing mature microRNAs in the database. From the long list of predicted mature microRNAs we selected possible 82 microRNA sequences (Table 1) for further analysis based on microRNA structural prediction kinetics and binding to potential target sequences. Target gene prediction software miRU (Zhang, 2005), RNA22 (Hyunh et al., 2006) and RNAhybrid (Krüger & Rehmsmeier, 2006) were used to select microRNAs based on their potential target genes from the Arabidopsis genome based on microRNA complementarity. Using these criteria we selected 82 mature viral microRNA sequences to confirm their occurrence and level in virus infected plants. Microarray analysis was performed using these 82 sequences as reverse complimentary probes on RNA extracted from wild type (WT) Col-0 virus infected and control mock plants. The results revealed significantly elevated levels of several predicted viral microRNAs in virus-infected plants (Table 1). We selected two putative microRNAs, TuMV-mir-S1 and TuMV-mir-S2, one with a low and one with a high expression signal, respectively, and confirmed that these small RNAs could be detected by northern blot analysis on infected Col-0 plants (FIGS. 2 A-2B). Both of these microRNAs originate from negative strand of viral RNA. A previously reported protocol for DNA/RNA hybrid primer-based microRNA cloning (Lu et al., 2005) was then used to clone these two putative microRNAs TuMV-mir-S1 and TuMV-mir-S2. RNA from a dcl2 dcl3 dcl4 triple mutant (Fusaro et al., 2006; Brosnan et al., 2007) was also probed for detection of TuMV-mir-S1 and TuMV-mir-S2, and while the putative microRNAs were not detected, larger RNA species indicative of microRNA precursors were surprisingly detected in virus-infected plants (FIGS. 2 A-2B). These results suggested that one or more of these dicers were responsible for processing viral microRNA precursors into mature microRNAs. Probing dcl2 dcl3 dcl4 triple mutants for TuMV revealed the occurrence of decreased full length viral RNA accumulation in the triple mutant line at 14 dpi (FIG. 2C). This indicated that a decrease in microRNA level might have a negative effect on the virus level in plant cells.

Role of DICER-Like Proteins in TuMV microRNA Biogenesis

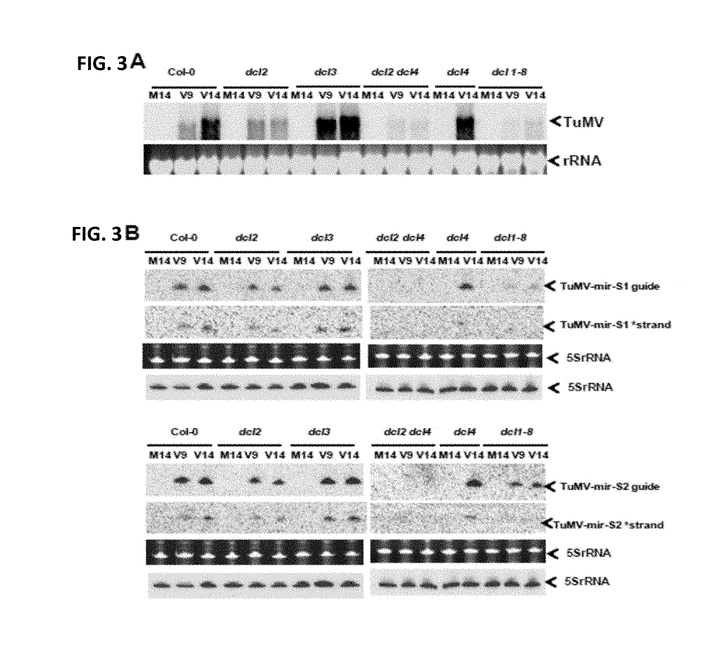

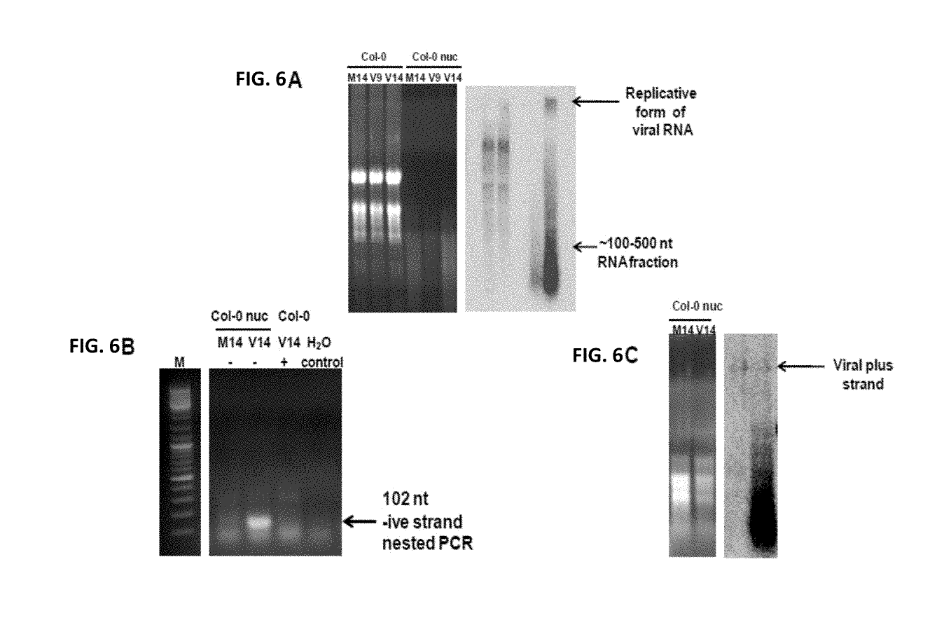

The role of Arabidopsis dicer proteins DCL2 and DCL4 in RNAi-based defence against some viruses has already been established. In these instances, DCL2 and DCL4 act redundantly and dcl2 dcl4 double mutants are particularly susceptible to the virus (Deleris et al., 2006). In contrast, at 14 dpi (days post inoculation) the level of full-length TuMV-BRS1 isolate RNA was considerably lower in the dcl2 dcl4 double mutant (FIG. 3A). As for the dcl2 dcl3 dcl4 triple mutant (FIGS. 2A-2B), probing the small RNA fraction from virus-infected and mock dcl2 dcl4 plants by northern blot hybridisation using antisense 21nt oligonucleotide probes failed to detect mature TuMV-mir-S1 and TuMV-mir-S2 (FIG. 3B). We could detect accumulation of a microRNA precursor sequence in virus-infected dcl2 dcl4 plants (FIG. 7A), albeit at lower levels than in wild type Col-0. This is consistent with the greatly diminished levels of the virus in the double mutant (FIG. 3A). The localisation of microRNA precursor in wild-type Col-0 and dcl2 and dcl4 single mutants was found to be in the nucleus (FIG. 7B). Thus TuMV accumulation correlates positively with the presence of mature viral microRNAs, contrary to the reported observations for viral siRNAs, where decrease in virus accumulation is associated with increased viral siRNAs in plants (Wang et al., 2010). Northern blot hybridisations were also used to determine the levels of TuMV-mir-S1 and TuMV-mir-S2 in small RNA fractions of dcl1-8 (Brosnan et al., 2007), dcl2-1, dcl3-1 and dcl4-2 (Brosnan et al., 2007) independent single mutants (FIG. 3B). The level of detected microRNAs was considerably lower in dcl1-8 and dcl2 plants compared to wild type. This reduced abundance of TuMV-mir-S1 and TuMV-mir-S2 in these two dicer mutant lines indicates that these plant proteins play important roles in the biogenesis of viral microRNAs. We also probed the TuMV-mir-S1* and TuMV-mir-S2* strands in all these lines and the level was considerably lower compared to the guide strand (FIG. 3B). This observation is in difference with the viral siRNA where more or less similar levels are detectable for both small RNA strands (Mlotshwa et al., 2008).

Our results are consistent with DCL1 producing the precursor from viral RNA in the nucleus, followed by further processing by DCL2 and DCL4 to produce the mature microRNAs. MicroRNA levels were significantly reduced in dcl2 plants compared to dcl4 plants, and therefore DCL2 is likely to be primarily responsible for the final cleavage to produce the mature microRNA. However, in the absence of DCL2, DCL4 compensated for the deficit (FIG. 3B). When cytoplasmic and nuclear RNA fractions from infected Col-0 plants were blotted and probed, we observed the presence of viral microRNA precursor in the nucleus, but not the mature microRNA in wild-type Col-0 (FIG. 3C). This could indicate that the final step in microRNA processing by DCL2 and DCL4 occurs in the cytoplasm. Alternatively, if final processing by DCL2 and DCL4 occurs in the nucleus, the mature microRNAs must be rapidly exported to the cytoplasm.

Consistent with the importance of DCL1, DCL2 and DCL4 in microRNA biogenesis, DCL3 appears to have an inhibitory effect on the level of viral microRNAs and viral replication because both of these were increased in the dcl3 mutant. Previous studies have revealed the role of DCL3 in the synthesis of longer (24 nt) microRNAs from the same microRNA precursors which are processed by DCL1 for the synthesis of 21-22 nt microRNAs in Arabidopsis (Vazquez et al., 2008). A simple explanation for these results is that DCL3 competes with the other DCLs for processing viral RNA, but that its 24 nt products are not used by AGO1 to execute microRNA-mediated silencing of endogenous Arabidopsis mRNA targets that contribute to viral defence. To address this possibility, we investigated the role of AGO1 in TuMV replication.

TuMV Replication is Severely Affected in ago1 Mutants

In plants AGO1, HYL1 and HASTY (HST) are of fundamental importance in endogenous microRNA biogenesis and action (Mallory & Bouche, 2008). Mutant lines for these genes, ago1-25 (Morel et al., 2002), hyl1-2 (SALK_064863) and hst-15 (SALK_079290) were therefore inoculated with TuMV to investigate the effect of these mutations on viral microRNA levels and viral replication. Interestingly, TuMV-mir-S1 and TuMV-mir-S2 were not detected and viral replication was insignificant in ago1-25 plants (FIG. 3D). This showed that AGO1 was absolutely required for TuMV infectivity and suggested that viral microRNAs may guide AGO1 to repress host target genes. Virus infectivity was also reduced in hst-15 plants but the virus level was considerably higher compared to ago1-25 plants (FIG. 7C). The results indicate that HST may be involved in export of the viral microRNA precursor from the nucleus to the cytoplasm. As the microRNA level was affected in hst-15 and the viral RNA was also considerably low, but not completely absent, this suggests that another exportin might also be involved in export of the viral microRNA in addition to HST. In hyl1-2 plants the virus level was similar to wild type plants at 14 dpi while it was considerably higher than in wild-type Arabidopsis plants at 9 dpi (FIG. 7C). HYL1 is one of five double stranded RNA binding proteins (DRBs) in Arabidopsis that associates with DCL1 and is involved in endogenous microRNA biogenesis. As for dcl3, hyl1 mutations may increase that flux of processed viral RNA through one of the other DRBs that are associated with DCL2 and/or DCL4 and more centrally involved in viral microRNA biogenesis.

### TuMV-mir-S1 Targets the Stress-Related Gene HVA22D

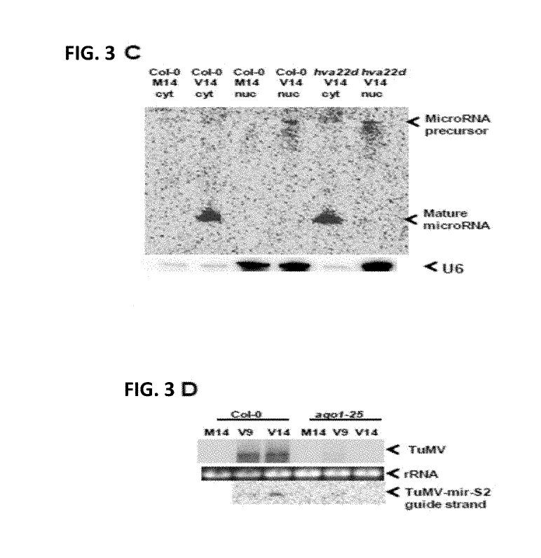

To search for likely target mRNAs for TuMV-mir-S1, HVA22d was selected based on sequence complementarity to TuMV-mir-S1 (FIG. 4A) and its reported role in plant stress response. HVA22d shows increased expression under cold, drought and unfavorable environmental conditions. This protein is induced by ABA and its yeast homologue, YOP1, has been found to regulate cellular vesicular trafficking in stressed cells (Brands & David Ho, 2002). Among the five members in the Arabidopsis gene family (HVA22a-HVA22e), HVA22d is induced by abscisic acid to the highest level in vegetative tissues (Chen et al., 2002), which is the major site for TuMV infection. HVA22d RNAi Arabidopsis plants contain elevated levels of autophagy, and mutants are defective in floral development (Chen et al., 2009). A WRKY21 protein interacts with VP1 and ABI5 to act positively in ABA-mediated induction of HVA22 in creosote bush (Zou et al., 2004). These reports implicate HVA22 across multiple signalling pathways in Arabidopsis.

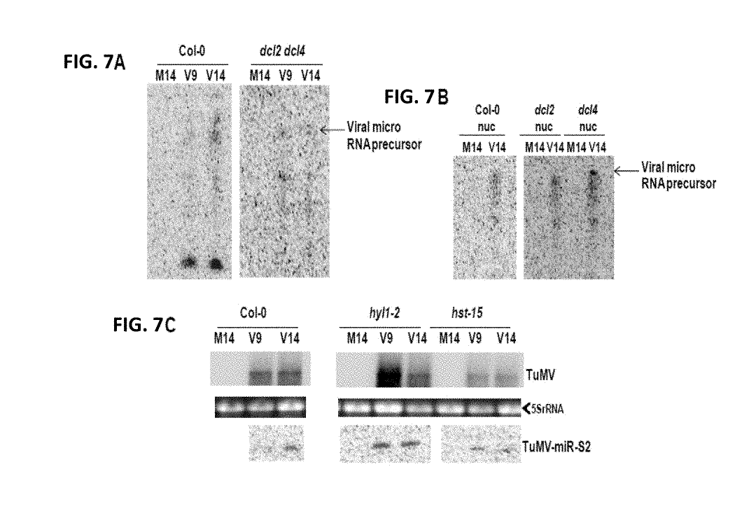

TuMV-mir-S1 could potentially bind to the target HVA22d transcript in two possible manners, both encompassing the stop codon and extending 15-18 nt downstream into the 3′ UTR (FIG. 4A). We initially developed a transient GFP-fused reporter system to demonstrate TuMV-mir-S1-guided cleavage of the HVA22d target site in Nicotiana benthamiana leaves. A 96 nucleotide sequence from HVA22d comprising the target sequence, along with a 43 nucleotide 5′ and a 27 nucleotide 3′ flanking region including the stop codon, was cloned downstream of a GFP reporter gene driven by the CaMV 35S promoter (FIG. 8A). The putative microRNA precursor sequence was also cloned for constitutive expression under the control of a 35S promoter in a separate T-DNA vector (FIG. 8A). The two constructs were co-agroinfiltrated in N. benthamiana leaves (Bendahmane et al., 1999) and GFP accumulation was visualised under a fluorescence microscope (FIG. 8B). Fluorescence intensities of infiltrated tissue showed that co-infiltration of the TuMV-mir-S 1 precursor transgene along with the GFP-HVA22d target transgene caused a marked decrease in GFP accumulation as compared to the control which was 35S-GFP construct co-agroinfiltrated with the precursor construct (FIG. 8B). Six independent homozygous transgenic Arabidopsis lines with the GFP-HVA22d transgene construct were also produced and one line was selected and inoculated with TuMV. GFP transcript levels were quantified at three different time intervals including 5 dpi, 9 dpi and 14 dpi. The level of GFP was unaffected at 5 and 9 dpi but was significantly reduced at 14 dpi (FIG. 4B) which correlates with the appearance of TuMV-mir-S1 in virus infected plants (FIGS. 2 and 3). Quantification of the transcript using GFP specific primers for qRT-PCR confirmed the results for the northern blot hybridisation (FIG. 4C).

To further verify the effect of HVA22d on virus replication and proliferation in Arabidopsis, an insertion mutant for hva22d (SALK_061029) was obtained from ABRC (Alonso et al., 2003). HVA22d has 3 introns and the T-DNA insertion was within the last exon in the region encompassing ˜42 nt upstream of stop codon and TuMV-mir-S1 binding site. Wild-type Col-0 and the homozygous insertion mutant plants were inoculated with TuMV and subjected to northern analysis to evaluate the level of virus. The results demonstrated a significant increase in the level of viral RNA in the hva22d insertion mutant plants compared to wild type (FIG. 4D). The role of this protein in virus resistance has not been previously demonstrated. Our results suggest that HVA22d, perhaps along with other signaling components, may play a fundamental role in plant defence against viruses.

### Discussion

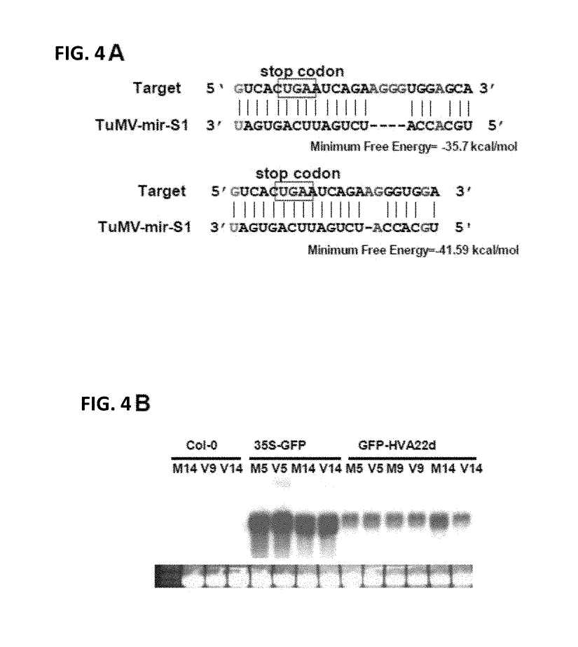

The results show that our BRS1 isolate of TuMV encodes a microRNA that targets the viral defence gene HVA22d. Production of the active microRNA requires the combined action of DCL1, DCL2 or DCL4 and AGO1 (FIGS. 2-4). A model for the biogenesis of this microRNA is shown in FIG. 5. The microRNA exportin HASTY is also required for efficient amplification of this virus in Arabidopsis plants (FIG. 7C). While it has been generally assumed that viral RNAs are restricted to the cytoplasm, our study has revealed that this virus not only moves into the host cell nucleus but the replication intermediate is also found to be localised mainly in the nucleus. This compartmentalisation of the replicating virus may protect it from microRNA-mediated degradation in the cytoplasm, but it also allows the DCL1-mediated first step in microRNA biogenesis to take place in the nucleus. It is particularly surprising, however, that maturation of the microRNA is mediated by DCL2 and DCL4 (FIG. 2), as these dicers protect Arabidopsis from other strains of viruses via RNAi-mediated viral degradation. Thus, our isolate of TuMV has recruited components of the host RNAi machinery that normally produces siRNAs against viruses, to produce a microRNA that targets a plant defence gene. The presence of mature microRNA in the cytoplasm but not the nucleus suggests that the DCL2/DCL4 mediated microRNA maturation step occurs predominantly in the cytoplasm (FIG. 5). This is the first report that describes a role for HVA22d in virus resistance. The fact that this gene is involved in abiotic stress response (Brands & Ho, 2002; Chen et al., 2002; Chen et al., 2009; Zou et al., 2004) provides further evidence for cross-talk between stress and virus resistance pathways. The role of abscisic acid in the plant's pathogen defence response is quite diverse. Elevated ABA levels confer increased resistance to the fungal necrotrophic pathogen Alternaria brassicicola. In contrast, the ABA biosynthetic mutants have decreased susceptibility to the bacterial pathogen Pseudomonas syringae, the oomycete Hyaloperonospora and the fungus Fusarium oxysporum in Arabidopsis (Fan et al., 2009; Anderson et al., 2004). A role of ABA in pathogen defence by inducing increased callose deposition has also been established (Bruce et al., 2007). These findings are supported by the fact that Chitosan (CHT, 2-amino-2-deoxy-b-D-glucosamine) activates Ca′ dependent callose synthase and elevates ABA levels, resulting in resistance to Tobacco necrosis virus (Iriti & Faoro, 2008). Callose deposition imparts partial resistance to the virus, possibly by impairment of cell-to-cell movement of the virus through plasmodesmata.

We found two overlapping potential target sites for the TuMV-mir-S1 microRNA in HVA22d (FIG. 4A). One possible microRNA-target complex has a single mismatch and a bulge in the target sequence after the seed region, and the other has two mismatches and a bulge corresponding to the 5′ half of the microRNA. Cleavage of targets by several endogenous microRNAs are reported to be sensitive to mismatches, particularly involving the 5′ half of the microRNA (Schwab et al., 2005; Palatnik et al., 2007), and yet, lower levels of a GFP transgene with a HVA22d target were observed upon inoculation with the virus. Our results indicate that cleavage of the target can occur with some mismatches in the 5′ half of the microRNA. The detection of microRNAs encoded by a plant RNA virus reveals the existence of a conserved mechanism between plants and animals. The discovery of 17 nucleotide unusually small RNAs (usRNAs) derived from Kaposi sarcoma associated herpesvirus K12-1 microRNA emphasize the significance of viral microRNAs and their proficiency in gene regulation even after partial degradation (Li et al., 2009). There are likely to be more microRNAs derived from our isolate of TuMV which could potentially target other genes (Table 1). Based on our research, it also appears likely that plant viral-encoded microRNAs may be common and represent an added level of complexity in plant-virus interactions. The detection of a microRNA in a plant virus that targets host defence genes opens up a new area of research in plant virus interactions. It should provide new insights into how viruses so successfully infect plants in the face of complex plant defence mechanisms. In addition, knowledge of microRNAs in plant viruses could also aid in controlling viral infection in plant species of economic importance.

### Example 2: Identification of microRNAs from Other Plant Viruses and Identification of their Host Target Genes

### Project Aims

1. Tomato spotted wilt virus (ToSWV) and tobacco mosaic virus (TMV) sequence search and selection from Genbank.
2. Selected sequences were subjected to miRNA precursor prediction software analysis.
3. Search predicted precursors and look for potential mature miRNAs.
4. Target search for predicted mature miRNAs in Arabidopsis and Tomato.
5. Cloning of over expression constructs of predicted miRNA precursors.
6. Cloning of target-GFP fusion constructs for potential target sequences.
7. Transient analysis through co-infiltration in Nicotiana benthamiana leaves.
8. Site directed mutagenesis of target sequences as a resistance development strategy.

### Materials and Methods

### Cloning Strategy

Top 10 E. coli chemically competent cells were used for transformation. The transformation in E. coli was done by the heat shock method, while Agrobacterium transformation was carried out by electroporation.

Precursor sequences were cloned in a binary vector using the 35S promoter and the 35S terminator. The sequence was cloned using HindIII and EcoRI as restriction enzymes for cloning.

Target sequences were cloned in a pUC18 based bacterial cloning vector pUC18-GFPST-sp using SalI and PstI as the cloning enzymes. The cassette with 35S promoter-GFP-target sequence-terminator was then lifted in binary vector pGreen0229 using EcoRI for cloning.

### Transformation Protocol Using Heat Shock

1. Turn on water bath to 42° C.
2. Put competent cells in a 1.5 ml tube. For transforming of a DNA construct, use 50 μl of competent cells. For transforming a ligation, use 100 μl of competent cells.
3. Keep tubes on ice.
4. Add 50 ng of circular DNA to E. coli cells. Incubate on ice for 10 min to thaw competent cells.
5. Put tube with DNA and E. coli into water bath at 42° C. for 90 seconds.
6. Put tubes back on ice for 2 minutes to reduce damage to the E. coli cells.
7. Add 1 ml of LB (with no antibiotic added). Incubate tubes for 1 hour at 37° C.
8. Spread about 100 μl of the resulting culture on one LB plate (with appropriate antibiotic) and centrifuge the remaining culture; discard all the supernatant leaving only 100 μl of media. 9. Resuspend the cells and spread on another plate with antibiotic. Grow overnight at 37° C.
10. Pick colonies about 12-16 hours later and screen for required clones.
Agroinfiltration of Nicotiana benthamiana Leaves

Agroinfiltration experiments were performed on N. benthamiana. N. benthamiana seeds were planted and grown in a growth chamber at 26° C. under a 16 hour light and 8 hour dark photoperiod. Plants were grown for 5 weeks before infiltration. Transformed A. tumefaciens (strain GV3101) pure cultures were grown from a single colony in a shaker for 2 days at 28° C. and 200 rpm in 5 ml LB medium (1% tryptone, 1% yeast extract, and 0.5% NaCl) containing 25 mg/l rifampicin 10 mg/l tetracyclin and 50 mg/l kanamycin to select for transformed Agrobacterium cells. We used 100 μl of this preculture to inoculate 10 ml of LB with all the three antibiotics. The culture was grown overnight and cells were harvested by centrifugation at 4500 rpm for 10 minutes. The pellet was then resuspended in 10 mM MgCl₂ to an OD₆₀₀ of 1 and acetosyringone to a final concentration of 200 μm was added to the cells. The resuspended cells were left at room temperature for 4-5 hours. The microRNA precursor overexpression construct was mixed along with the microRNA target-GFP construct in a ratio of 3:1, respectively.

We infiltrated N. benthamiana leaves on the back (abaxially) using a 5 ml syringe. For infiltration we pressed the mouth of the syringe without a needle, on the leaf where branching of veins was visible. A finger was kept on the other side of the leaf for support. A single plant was infiltrated in 4-5 leaves and we infiltrated 2-3 spots per leaf. GFP was used as a visual marker. The GFP expression was monitored visually under a fluorescence microscope after 3 days.

### Results

Custom DNA synthesis of ˜200 nt miRNA precursors was obtained. Cloning of six precursor over expression constructs (four from ToSWV, two from TMV) in plant gene expression vector downstream of the CaMV 35S promoter.

miRNA precursors were selected from ToSWV genome sequence from the following regions:
1. RNA polymerase
2. Nonstructural protein
3. Intergenic region
4. N gene (nucleocapsid protein)

miRNA precursors were selected from TMV genome sequence from the following regions:
1. Replication protein
2. 3′UTR

Cloning and sequence confirmation was successfully carried out for nine potential target sequences of ˜200 nt each, including the predicted miRNA binding sites. These were cloned downstream of GFP and all nine target sequence-GFP fusion cassettes were subcloned in pGreen0229 for plant expression.

Viral miRNA target genes for GFP fusion constructs were predicted for the following genes:
1. Pathogen-related protein 5 (PR5)
2. Lectin protein kinase (Lec)
3. Lesion inducing protein (HR ind)
4. Vanguard 1 (VGD1)
5. Tombusvirus replication protein 1 (Tom1)
6. NRPD1B
7. Expansin8 (EXP8)
8. Brassinosteroid signalling regulator (BEH1)
9. Brassinosteroid signalling regulator (ATBS1)

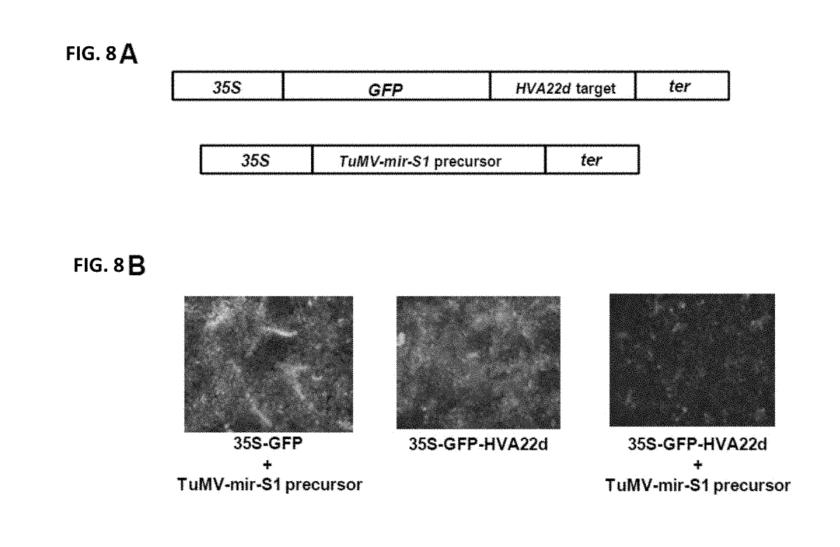

Agro-infiltration of ToSWV miRNA precursor constructs along with their respective target sequence clones was carried out in N. benthamiana leaves. Target genes with differential GFP fluorescence as observed through microscopy was confirmed experimentally in planta for NRPD1B, PR5, BEH1 and EXP8 (FIGS. 9A-9D). Primer designing and synthesis for primer extension was based on site directed mutagenesis.

### Example 3: miRNA Precursor Prediction in Several Other Viruses

Fiji disease virus, Tobacco streak virus Isolate okra and Tobacco etch virus were subjected to miRNA precursor prediction with miRNAfinder and findmiRNA (Adai et al., Genome Research 15:78-91, 2005) with strong predictions of miRNA precursor sequences made for these viruses (Table 3).

### Example 4: Prevention of Viral miRNA Silencing

The miRNA binding site for the viral defence gene HVA22d was mutated via a silent mutation to examine the ability of viral miRNA to silence a host target gene that had been mutated.

### Materials and Methods

### Cloning

Non-mutated HVA22d target sequence:
(SEQ ID NO: 88)
CTCACAGTCACTGAATCAGAA.
Mutated HVA22d target sequence:
(SEQ ID NO: 89)
CTCACAGCCATTAGCCACATA.

Both the non-mutated and mutated HVA22d target sequences were fused to GFP.

Transformation/electroporation of Agrobacterium tumefaciens
1. Electroporation cuvettes 1 mm gap were placed on ice
2. Thaw competent cells on ice (50 μl per transformation).
3. Added plasmid DNA (1 μl of E. coli miniprep) to the cells, and mix them together on ice.
4. Transferred the mixture to the pre-chilled electroporation cuvette. Carried out electroporation as recommended for E. coli by the manufacturer of the chosen electroporator.
5. We used the Bio-Rad electroporator with a 1-mm cuvette, using the following conditions:
   Capacitance: 25 μF
   Voltage: 1.44 kV
   Resistance: 129 S2
   Pulse length: 5 msec
6 Immediately after electroporation, add 1 ml of LB to the cuvette, and transfer the bacterial suspension to a 1.5 ml tube. Incubate for 1 hour at 28° C. with gentle agitation.
7. Spread 50 μl of the cells on an LB agar plate containing the Kanamycin, Rifampicin and Tetracyclin.
8. Incubated the plates for 2-3 days at 28° C.
9. Inoculated 5 ml liquid cultures with single colonies from the plates.
10. Culture tubes were kept at 28° C. on shaker for 48 hours with vigorous shaking (˜200 rpm).
11. PCR was carried out to verify the presence of plasmid DNA.

### Results

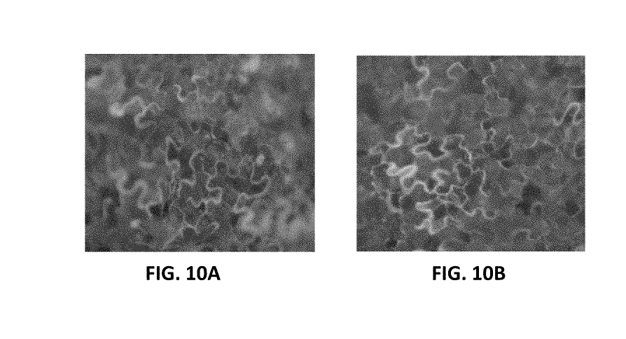

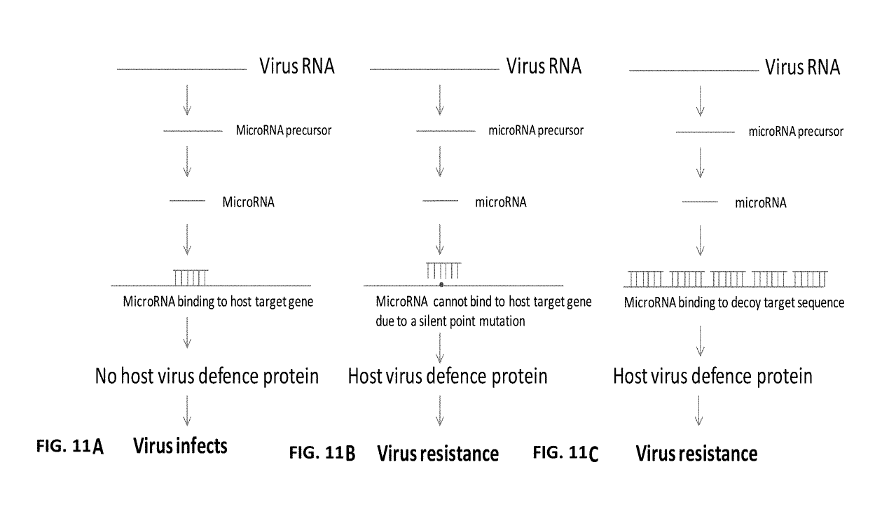

Viral miRNA had no effect on silencing if the host HVA22d target gene was mutated (FIG. 10). These results demonstrate that the addition of miRNA does not lead to silencing of the target host gene if a silent point mutation is introduced in the miRNA binding site (see, FIGS. 11A-11B). Hence, the miRNA has no visible effect on gene expression and the expression of the GFP fusion is no longer compromised by the presence of viral miRNA.

### Example 5: Decoy Sequences for Capture of Viral miRNAs

As will be understood by one of skill in the art, the discovery of a new class of small plant virus RNA molecules involved in modulating a plant defence response enables the creation of decoy target sequences, which, when introduced into a plant (including plant parts), reduce, decrease, or mitigate the susceptibility of the plant to a pathogen. Such decoy target sequences are capable of binding, annealing to, hybridising to, or otherwise recognising and capturing plant viral miRNAs, including one or more of the isolated plant viral miRNAs of the invention.

An example of a decoy sequence to capture TuMV miRNA is:

(SEQ ID NO: 90)
ATCACTGAATCAGATGGTGCA

The sequence can include 15-20 repeats of the decoy sequence (equal to 315-420 bp), but the sequence could also be longer or shorter. Unlike the target sequence in HVA22d, the above exemplary decoy sequence is a perfect match to the viral sequence, and will have a much stronger affinity to the miRNA than HVA22d. When expressed by a strong constitutive (or plant defence-inducible) promoter in plants, enough decoy transcripts will be present to very effectively capture viral RNAs, leaving plant defence transcripts generally unaffected (see, FIGS. 11A and 11C).

Preferably, combined constructs are made, where several potential targets to viral miRNAs against one or several viruses (or virus strains) are constructed. The combined effect will be even stronger and should provide broad protection against multiple isolates and/or different viruses that affect the same plant.

TABLE 1
Predicted viral microRNAs and antisense viral microRNA microarray probe sequences
(SEQ ID NO: 1-82 and their complements SEQ ID NOS: 139-220, respectively.

| SEQ | | | Predicted mature | Antisense microRNA |
|---|---|---|---|---|
| ID NO: | Probe name | MicroRNA name | microRNA sequence | probe sequence |
| 1 | TuMV miR 1 | TuMV-miR-S1 | UGCACCAUCUGAUUCAGUGAU | AUCACUGAAUCAGAUGGUGCA |
| 2 | TuMV miR 2 | TuMV-miR-S3 | GCGAGUUCCCAUUCUAUCUUCU | AGAAGAUAGAAUGGGAACUCGC |
| 3 | TuMV miR 3 | TuMV-miR-S2 | GUUGAGUGCUUGGUGGUACAC | GUGUACCACCAAGCACUCAAC |
| 4 | TuMV miR 4 | TuMV-miR-S4 | UGACUUUGUCAUGUGUGUUGU | ACAACACACAUGACAAAGUCA |
| 5 | TuMV miR 5 | TuMV-miR-S5 | UAAAGCCUUGCCUGUUUUGUU | AACAAAACAGGCAAGGCUUUA |
| 6 | TuMV miR 6 | TuMV-miR-S6 | AAAACAUUGAUCACAAGAGAU | AUCUCUUGUGAUCAAUGUUUU |
| 7 | TuMV miR 7 | TuMV-miR-S7 | GGAAUGUGGGUGAUGAUGGAU | AUCCAUCAUCACCCACAUUCC |
| 8 | TuMV miR 8 | TuMV-miR-S8 | ACGUUGGGUGAACACUCAGCAA | UUGCUGAGUGUUCACCCAACGU |
| 9 | TuMV miR 9 | TuMV-miR-S9 | GUUGGUGGUAAAGUGUCUAGUA | UACUAGACACUUUACCACCAAC |
| 10 | TuMV miR 10 | TuMV-miR-S10 | UCCAAAUGAUUUUGCUGAGAAAU | AUUUCUCAGCAAAAUCAUUUGGA |
| 11 | TuMV miR 11 | TuMV-miR-S11 | CAAUAGCGUGUCUUGGGUUGGU | ACCAACCCAAGACACGCUAUUG |
| 12 | TuMV miR 12 | TuMV-miR-S12 | UGAUGGAUGGUGACGAUCAGG | CCUGAUCGUCACCAUCCAUCA |
| 13 | TuMV miR 13 | TuMV-miR-S13 | AAUAUAAACGGAAUGUGGGUG | CACCCACAUUCCGUUUAUAUU |
| 14 | TuMV miR 14 | TuMV-miR-S14 | AACGGAAUGUGGGUGAUGAUGGA | UCCAUCAUCACCCACAUUCCGUU |
| 15 | TuMV miR 15 | TuMV-miR-S15 | UUUAACCGACAUGAGCCUAGCUC | GAGCUAGGCUCAUGUCGGUUAAA |
| 16 | TuMV miR 16 | TuMV-miR-S16 | AUGCAUUUGAUUUCUAUGAAAUG | CAUUUCAUAGAAAUCAAAUGCAU |
| 17 | TuMV miR 17 | TuMV-miR-S17 | AUUUCUAUGAAAUGACUUCUAG | CUAGAAGUCAUUUCAUAGAAAU |
| 18 | TuMV miR 18 | TuMV-miR-S18 | GUCGAGGCUAGGGCUAAUAUCA | UGAUAUUAGCCCUAGCCUCGAC |
| 19 | TuMV miR 19 | TuMV-miR-S19 | AUUUUAUUGGUGUUAGCGCAU | AUGCGCUAACACCAAUAAAAU |
| 20 | TuMV miR 20 | TuMV-miR-S20 | CGAAAGCUAUACAACCAGGAG | CUCCUGGUUGUAUAGCUUUCG |
| 21 | TuMV miR 21 | TuMV-miR-S21 | ACCAGGAGUAGUAUGUGCUGG | CCAGCACAUACUACUCCUGGU |
| 22 | TuMV miR 22 | TuMV-miR-S22 | GCUUCCUUGCAUAUCGCAGUAG | CUACUGCGAUAUGCAAGGAAGC |
| 23 | TuMV miR 23 | TuMV-miR-S23 | UUGCAUAUCGCAGUAGUGAUC | GAUCACUACUGCGAUAUGCAA |
| 24 | TuMV miR 24 | TuMV-miR-S24 | GAAUGGGUCAAGCACUGGAAGU | ACUUCCAGUGCUUGACCCAUUC |
| 25 | TuMV miR 25 | TuMV-miR-S25 | AUAAUAUGAAGGUCACGAAC | GUUCGUGACCUUCAUAUUAU |
| 26 | TuMV miR 26 | TuMV-miR-S26 | GCACAUGAAUGGGUCAAGCACU | AGUGCUUGACCCAUUCAUGUGC |
| 27 | TuMV miR 27 | TuMV-miR-S27 | UGAUGGAUGGUGACGAUCAGG | CCUGAUCGUCACCAUCCAUCA |
| 28 | TuMV miR 28 | TuMV-miR-S28 | UGGUGACGAUCAGGUGGAAUU | AAUUCCACCUGAUCGUCACCA |
| 29 | TuMV miR 29 | TuMV-miR-S29 | AUCGCACGCCUUUGUAAUUAGA | UCUAAUUACAAAGGCGUGCGAU |
| 30 | TuMV miR 30 | TuMV-miR-S30 | GAAGUCCAUCGCACGCCUUUGU | ACAAAGGCGUGCGAUGGACUUC |
| 31 | TuMV miR 31 | TuMV-miR-S31 | AAGAAUUGAAGAAUUUGACUU | AAGUCAAAUUCUUCAAUUCUU |
| 32 | TuMV miR 32 | TuMV-miR-S32 | UCAAAUUCUUCAAUUCUUGCUC | GAGCAAGAAUUGAAGAAUUUGA |
| 33 | TuMV miR 33 | TuMV-miR-S33 | UUGAAGAAUUUGACUUUGUUAU | AUAACAAAGUCAAAUUCUUCAA |
| 34 | TuMV miR 34 | TuMV-miR-S34 | UCGGGAAUUCCACCUGAUCGUC | GACGAUCAGGUGGAAUUCCCGA |
| 35 | TuMV miR 35 | TuMV-miR-S35 | CUGCCUAAAUGUGGGUUUGGCG | CGCCAAACCCACAUUUAGGCAG |
| 36 | TuMV miR 36 | TuMV-miR-S36 | GGUGUUAAAUCUACCUUUAAAGC | GCUUUAAAGGUAGAUUUAACACC |
| 37 | TuMV miR 37 | TuMV-miR-S37 | AAGAGGCAUGUGUGGUGUUAA | UUAACACCACACAUGCCUCUU |
| 38 | TuMV miR 38 | TuMV-miR-S38 | AUCGAACUGUGAUCCAUCUGCG | CGCAGAUGGAUCACAGUUCGAU |
| 39 | TuMV miR 39 | TuMV-miR-S39 | AUAGUGAACUAUCGAACUGUGA | UCACAGUUCGAUAGUUCACUAU |
| 40 | TuMV miR 40 | TuMV-miR-S40 | CCAUGAAUUCUAAUCGGAUGUUG | CAACAUCCGAUUAGAAUUCAUGG |
| 41 | TuMV miR 41 | TuMV-miR-S41 | AUCGGAUGUUGAGUACUGCGU | ACGCAGUACUCAACAUCCGAU |
| 42 | TuMV miR 42 | TuMV-miR-S42 | AACUGUGAUCCAUCUGCGUCG | CGACGCAGAUGGAUCACAGUU |
| 43 | TuMV miR 43 | TuMV-miR-S43 | AAUCAACAUCCAACACUCGAU | AUCGAGUGUUGGAUGUUGAUU |
| 44 | TuMV miR 44 | TuMV-miR-S44 | AUUAGCACUAUGGGUCAGAAU | AUUCUGACCCAUAGUGCUAAU |
| 45 | TuMV miR 45 | TuMV-miR-S45 | UGCGAGUUCCCAUUCUAUCUUCU | AGAAGAUAGAAUGGGAACUCGCA |
| 46 | TuMV miR 46 | TuMV-miR-S46 | AAUCAACAUCCAACACUCGAUG | CAUCGAGUGUUGGAUGUUGAUU |
| 47 | TuMV miR 47 | TuMV-miR-S47 | GGUGAGAGUAGGGCGUAUAGU | ACUAUACGCCCUACUCUCACC |
| 48 | TuMV miR 48 | TuMV-miR-S48 | GGAACCAAUUGGAAGUCACUGUU | AACAGUGACUUCCAAUUGGUUCC |
| 49 | TuMV miR 49 | TuMV-miR-S49 | AUUUGGGAUGCUCUGCAUUGAG | CUCAAUGCAGAGCAUCCCAAAU |
| 50 | TuMV miR 50 | TuMV-miR-S50 | UAUUCUGCUUCUCUUUCCUCA | UGAGGAAAGAGAAGCAGAAUA |
| 51 | TuMV miR 51 | TuMV-miR-S51 | AAGAAGAGGAACCAAUUGGAAGU | ACUUCCAAUUGGUUCCUCUUCUU |
| 52 | TuMV miR 52 | TuMV-miR-S52 | GCUCUGCAUUGAGGAAACUGA | UCAGUUUCCUCAAUGCAGAGC |
| 53 | TuMV miR 53 | TuMV-miR-S53 | AUUGAGGAAACUGAAGAAGAGG | CCUCUUCUUCAGUUUCCUCAAU |
| 54 | TuMV miR 54 | TuMV-miR-S54 | UUGCAGUGCUUGCGGUUCGAG | CUCGAACCGCAAGCACUGCAA |
| 55 | TuMV miR 55 | TuMV-miR-S55 | UGCGUGUACCUGUGGAUGCAUU | AAUGCAUCCACAGGUACACGCA |
| 56 | TuMV miR 56 | TuMV-miR-S56 | UAUCUCACCACUUGACUUGUGU | ACACAAGUCAAGUGGUGAGAUA |
| 57 | TuMV miR 57 | TuMV-miR-S57 | CGUGUGCUCUCGAUCACUACUGC | GCAGUAGUGAUCGAGAGCACACG |
| 58 | TuMV miR 58 | TuMV-miR-S58 | GGAAGCACCUAUCAAAGCCUU | AAGGCUUUGAUAGGUGCUUCC |
| 59 | TuMV miR 59 | TuMV-miR-S59 | GGUGGUGGUGUUGGUGAUAGCU | AGCUAUCACCAACACCACCACC |
| 60 | TuMV miR 60 | TuMV-miR-S60 | AGUGCUGGUUUGUUGGUGGUGG | CCACCACCAACAAACCAGCACU |
| 61 | TuMV miR 61 | TuMV-miR-S61 | GGUGAUAAACACACACUUCAGUA | UACUGAAGUGUGUGUUUAUCACC |
| 62 | TuMV miR 62 | TuMV-miR-S62 | AUGUUGAGUACUGCGUUGAUU | AAUCAACGCAGUACUCAACAU |
| 63 | TuMV miR 63 | TuMV-miR-S63 | AUCGAACUGUGAUCCAUCUGCG | CGCAGAUGGAUCACAGUUCGAU |
| 64 | TuMV miR 64 | TuMV-miR-S64 | AUAGUGAACUAUCGAACUGUGA | UCACAGUUCGAUAGUUCACUAU |
| 65 | TuMV miR 65 | TuMV-miR-S65 | AUCCAUCUGCGUCGCAGUAAAUC | GAUUUACUGCGACGCAGAUGGAU |
| 66 | TuMV miR 66 | TuMV-miR-S66 | GUUGGUGGUAAAGUGUCUAGUA | UACUAGACACUUUACCACCAAC |
| 67 | TuMV miR 67 | TuMV-miR-S67 | AAUAUAAACGGAAUGUGGGUG | CACCCACAUUCCGUUUAUAUU |
| 68 | TuMV miR 68 | TuMV-miR-S68 | GCUUUUCCAAAUGAUUUUGCUG | CAGCAAAAUCAUUUGGAAAAGC |
| 69 | TuMV miR 69 | TuMV-miR-S69 | UCGCCAUAUUUAAUCAACGCA | UGCGUUGAUUAAAUAUGGCGA |
| 70 | TuMV miR 70 | TuMV-miR-S70 | AACAGAGCAAGAAUUGAAGAA | UUCUUCAAUUCUUGCUCUGUU |
| 71 | TuMV miR 71 | TuMV-miR-S71 | AAUAUAACAAAGUCAAAUUCU | AGAAUUUGACUUUGUUAUAUU |
| 72 | TuMV-miR-S72 | TuMV-miR-S72 | AGUGGAACAAAACAUUGAUCA | UGAUCAAUGUUUUGUUCCACU |
| 73 | TuMV-miR-S73 | TuMV-miR-S73 | GGAGUUCUAGGAGGUGGAAUUU | AAAUUCCACCUCCUAGAACUCC |
| 74 | TuMV-miR-S74 | TuMV-miR-S74 | AUUCCACCUCCUAGAACUCCA | UGGAGUUCUAGGAGGUGGAAU |
| 75 | TuMV-miR-S75 | TuMV-miR-S75 | UAGACACCAUGGCAGACAAUUU | AAAUUGUCUGCCAUGGUGUCUA |
| 76 | TuMV-miR-S76 | TuMV-miR-S76 | AUUAGAUUCUUUGUUAAUGGCG | CGCCAUUAACAAAGAAUCUAAU |
| 77 | TuMV-miR-S77 | TuMV-miR-S77 | CUUUGUUAAUGGCGAUGAUCUG | CAGAUCAUCGCCAUUAACAAAG |
| 78 | TuMV-miR-S78 | TuMV-miR-S78 | AUGGGUAGAGAAGUUUAUGGG | CCCAUAAACUUCUCUACCCAU |
| 79 | TuMV-miR-S79 | TuMV-miR-S79 | UGACGACACCAUAGAACACUUC | GAAGUGUUCUAUGGUGUCGUCA |
| 80 | TuMV-miR-S80 | TuMV-miR-S80 | GAGAAGUUUAUGGGGAUGACG | CGUCAUCCCCAUAAACUUCUC |
| 81 | TuMV-miR-S81 | TuMV-miR-S81 | GCUUGCAGUCUCCCAAAACUGA | UCAGUUUUGGGAGACUGCAAGC |
| 82 | TuMV-miR-S82 | TuMV-miR-S82 | UCGUCUUCAUAGUCUUCGAAU | AUUCGAAGACUAUGAAGACGA |

TABLE 2
Average signal in microarray detection of predicted viral microRNAs in two
biological replicates.

| | S04 - Col-0 WT 1 | | S05 - Col-0 WT 2 | |
|---|---|---|---|---|
| MicroRNA | Averaged | S03 - Col-0 TuMV 1 | Averaged | S06 - Col-0 TuMV 2 |
| name | signal | Averaged signal | signal | Averaged signal |
| TuMV-miR-S1 | 20.32649846 | 71.05591952 | 16.532617 | 65.61584725 |
| TuMV-miR-S2 | 19.33101503 | 151.247494 | 23.34199454 | 143.4784947 |
| TuMV-miR-S3 | 14.9052225 | 23.6496207 | 14.03172058 | 23.51262288 |
| TuMV-miR-S4 | 14.08433167 | 18.12303173 | 8.846332746 | 16.40314444 |
| TuMV-miR-S5 | 16.35886103 | 29.48699287 | 7.701421089 | 31.76169172 |
| TuMV-miR-S6 | 11.42964635 | 29.31392461 | 5.421535241 | 21.09697769 |
| TuMV-miR-S7 | 23.78342837 | 194.2182135 | 36.39996752 | 194.5706369 |
| TuMV-miR-S8 | 9.250213197 | 24.01341911 | 9.730902729 | 28.40113939 |
| TuMV-miR-S9 | 17.14211854 | 344.537249 | 54.85285106 | 321.521866 |
| TuMV-miR-S10 | 12.27611523 | 24.4370101 | 18.74424464 | 21.57290686 |
| TuMV-miR-S11 | 15.98871658 | 247.1963702 | 31.34289794 | 242.1475478 |
| TuMV-miR-S12 | 28.00664147 | 156.0002421 | 35.25837463 | 155.1721301 |
| TuMV-miR-S13 | 19.67237988 | 141.3693399 | 19.63779414 | 127.7169006 |
| TuMV-miR-S14 | 18.97993439 | 214.2348405 | 16.75304359 | 196.9563673 |
| TuMV-miR-S15 | 14.61780566 | 72.85494565 | 14.46345171 | 59.92981846 |
| TuMV-miR-S16 | 13.33116986 | 7.732429262 | 4.993389149 | 2.466283408 |
| TuMV-miR-S17 | 8.580204855 | 6.458696522 | 7.331614317 | 2.55619786 |
| TuMV-miR-S18 | 23.08822614 | 128.8217504 | 30.88050067 | 128.1938779 |
| TuMV-miR-S19 | 13.06671984 | 10.25805895 | 5.809196047 | 6.365582326 |
| TuMV-miR-S20 | 20.68201753 | 193.1780031 | 25.15610219 | 159.7776271 |
| TuMV-miR-S21 | 26.42813404 | 380.5381636 | 9.884530051 | 324.3071275 |
| TuMV-miR-S22 | 12.53436707 | 24.76898893 | 11.99147661 | 19.93452686 |
| TuMV-miR-S23 | 17.32856584 | 50.80043611 | 6.320846575 | 44.77963309 |
| TuMV-miR-S24 | 18.42534512 | 258.8702381 | 22.92701547 | 218.151907 |
| TuMV-miR-S25 | 10.87786955 | 8.628719012 | 4.69590133 | 3.281789818 |
| TuMV-miR-S26 | 16.36500963 | 173.172826 | 40.09874403 | 152.7177717 |
| TuMV-miR-S27 | 18.70062211 | 148.2873201 | 44.31388785 | 153.8712864 |
| TuMV-miR-S28 | 18.69126253 | 101.9279654 | 27.86363139 | 97.66951333 |
| TuMV-miR-S29 | 16.43911785 | 112.2090385 | 16.42077024 | 79.43303419 |
| TuMV-miR-S30 | 21.48732695 | 56.26633508 | 34.1385499 | 68.89603162 |
| TuMV-miR-S31 | 7.423505223 | 7.206423163 | 6.218391992 | 1.52341963 |
| TuMV-miR-S32 | 13.02928297 | 6.774506581 | 8.476661846 | 1.914676302 |
| TuMV-miR-S33 | 13.99916362 | 7.000493881 | 8.370435952 | 0.72555799 |
| TuMV-miR-S34 | 23.01976525 | 38.55455053 | 29.99868114 | 33.61174519 |
| TuMV-miR-S35 | 13.57628869 | 64.72942743 | 31.46374721 | 64.03819169 |
| TuMV-miR-S36 | 12.93154024 | 7.826868534 | 14.94206957 | 4.579003951 |
| TuMV-miR-S37 | 14.13685742 | 184.7699558 | 24.12734159 | 153.0262467 |
| TuMV-miR-S38 | 13.44873781 | 43.68995819 | 13.85839487 | 36.16533702 |
| TuMV-miR-S39 | 15.54410594 | 56.70060367 | 13.47164203 | 52.73091103 |
| TuMV-miR-S40 | 10.05566329 | 17.98677598 | 6.578344242 | 15.39202841 |
| TuMV-miR-S41 | 20.65092178 | 132.6028825 | 12.3808827 | 130.9549457 |
| TuMV-miR-S42 | 10.13970155 | 38.74532414 | 12.05303707 | 26.87082182 |
| TuMV-miR-S43 | 12.98385878 | 41.0662419 | 18.11915056 | 45.0880652 |
| TuMV-miR-S44 | 20.1576251 | 308.1279932 | 35.03161789 | 320.9886851 |
| TuMV-miR-S45 | 13.91867221 | 24.58508503 | 20.23113937 | 24.75684499 |
| TuMV-miR-S46 | 11.17435667 | 39.69864231 | 25.07930083 | 42.36349965 |
| TuMV-miR-S47 | 19.01217513 | 333.9384162 | 30.54440232 | 310.0838017 |
| TuMV-miR-S48 | 12.95967212 | 23.452778 | 10.83033901 | 18.23340828 |
| TuMV-miR-S49 | 8.945650404 | 8.510196276 | 4.328108853 | 1.48007625 |
| TuMV-miR-S50 | 12.95644511 | 10.81345464 | 5.282344733 | 3.58614979 |
| TuMV-miR-S51 | 7.622080632 | 11.72864799 | 5.197999309 | 2.107313661 |
| TuMV-miR-S52 | 13.42594908 | 10.13552263 | 7.916099757 | 4.419497102 |
| TuMV-miR-S53 | 21.17435997 | 9.246764197 | 11.25872323 | 4.769787645 |
| TuMV-miR-S54 | 16.77512004 | 53.98741808 | 27.97830148 | 59.89958018 |
| TuMV-miR-S55 | 10.64026328 | 135.6653953 | 30.1636978 | 106.7083204 |
| TuMV-miR-S56 | 14.37865866 | 86.28422473 | 18.93438672 | 83.71910962 |
| TuMV-miR-S57 | 16.22924493 | 89.5161024 | 17.79567305 | 85.56151213 |
| TuMV-miR-S58 | 13.81803343 | 69.92178443 | 20.38438841 | 76.94899088 |
| TuMV-miR-S59 | 34.58434049 | 297.571708 | 36.13058499 | 273.1656021 |
| TuMV-miR-S60 | 33.29121196 | 623.0500904 | 35.93103918 | 631.3470325 |
| TuMV-miR-S61 | 11.24432396 | 27.60513371 | 20.19699368 | 26.34028906 |
| TuMV-miR-S62 | 12.53947284 | 157.8742465 | 26.90728784 | 129.693484 |
| TuMV-miR-S63 | 15.59833352 | 54.82178343 | 19.40274309 | 51.39323758 |
| TuMV-miR-S64 | 12.36393035 | 63.35475225 | 18.71001528 | 57.85559146 |
| TuMV-miR-S65 | 7.818317135 | 13.41212849 | 4.000695561 | 7.892684162 |
| TuMV-miR-S66 | 15.48387461 | 349.3151555 | 31.25583858 | 306.6172229 |
| TuMV-miR-S67 | 15.3433672 | 164.6013156 | 16.58441105 | 163.9162495 |
| TuMV-miR-S68 | 9.253344425 | 7.146300879 | 5.182431863 | 3.71610874 |
| TuMV-miR-S69 | 13.64587881 | 43.5872705 | 2.590757988 | 38.18244173 |
| TuMV-miR-S70 | 12.71940704 | 221.6082273 | 16.66877401 | 145.1072386 |
| TuMV-miR-S71 | 6.173498735 | 6.333241237 | 7.187789206 | 3.433985512 |
| TuMV-miR-S72 | 6.653365909 | 31.70463196 | 25.15964254 | 27.30100437 |
| TuMV-miR-S73 | 13.71954285 | 211.9232131 | 30.63015188 | 243.1320951 |
| TuMV-miR-S74 | 30.09064599 | 67.75627342 | 15.85891749 | 68.10986177 |
| TuMV-miR-S75 | 20.14148458 | 51.45882524 | 16.26487413 | 69.1017041 |
| TuMV-miR-S76 | 7.577817376 | 8.044787356 | 3.564182785 | 2.013210293 |
| TuMV-miR-S77 | 8.671305197 | 122.3723281 | 7.978111405 | 93.37914017 |
| TuMV-miR-S78 | 14.59390035 | 355.3362377 | 16.98874406 | 319.2430428 |
| TuMV-miR-S79 | 14.03116865 | 217.647409 | 34.46338112 | 180.7269152 |
| TuMV-miR-S80 | 14.65435619 | 281.3128188 | 45.84559896 | 258.7848414 |
| TuMV-miR-S81 | 11.51699966 | 5.947356324 | 13.12234364 | 3.823683708 |
| TuMV-miR-S82 | 15.4955477 | 25.34663488 | 9.585156702 | 28.78596111 |

Italic TuMV-mir-S1
Bold TuMV-mir-S2
Underline Other predicted viral microRNAs showing significant increase in level after virus infection.

TABLE 3
Predicted miRNA precursor
sequences from several viruses

Fiji disease virus
Segment 1
3242-3361
(SEQ ID NO: 91)
AACUUAAUUUUAAACGCACCACCUUAUCUUGGUGUUUAACAAAUUCU
ACAAACUUAAACAUGUGUUCUUCAUCGCUAGUAAAAAACAUCUGAUU
GAUAAGCAAGAAAGCGCCAUCUCCAU
2251-2360
(SEQ ID NO: 92)
CUACCUAAUUUAACUUUGUUUUUACGACGUAAAAUGAUUGAAGUAUU
AUAUGAACUUGACCCUACAGGCAAAUGUGCUAGAUAUUUCUUCGCUG
AAGAUAAAGAAUACUG
251-370
(SEQ ID NO: 93)
UUAGUUCUUUAGUCAAAGGUUCUGUUCCGAUGAAUAAAUCCCGGAUC
UUUUCUGCUUUAUUGAAAAUGGCAUCAAUCAUGUUAACUUUAUCAUU
AAGAAUUUUUUCAUACUCAGCAACUU
Segment 2
1636-1742
(SEQ ID NO: 94)
GAAUAGUCACAAAUUUGGUAAACAUUUGUAUGAUUUAAUGUCAGUAU
UUUGUAGGUCAGAACUGAUAGCGUAUGAAGCCAGGUAUGGAUGUUUU
AUUAAAUUUGAGA
2957-3076
(SEQ ID NO: 95)
GAUUGGUUUGUGGAUACUUUACUAGUAGUUCGAAUUCCCCUGAUGAC
UAUUUUAGUGUUGAUGAAGAUACUUUAUAUUUCAGUAUUGAUUUGGA
UGAACAUCCUGAAGUGUUUACGACCG
3011-3120
(SEQ ID NO: 96)
GUGUUGAUGAAGAUACUUUAUAUUUCAGUAUUGAUUUGGAUGAACAU
CCUGAAGUGUUUACGACCGUUGGCACAAAUGGAUUCAGUAUACAGUU
ACAAUUUAAGAAAGGA
2059-2178
(SEQ ID NO: 97)
AUGCAUUAAUAUUCCUAAAUUAUUAGCGGUGUUACCGAUAGUAUCUA
AGAUUAGAUCAUAUGCUUCUUUAGUUGAUGUUUUAGUAGGUUUAUGA
GUAAAAUAUCUCUGGCUUAGAUCUAU
Segment 3
3499-3608
(SEQ ID NO: 98)
AAUCCGUGUUAAAGACAAUUCUAGCUUUCUGAGAUUAUGAAGCUAUG
UUUUGGAGAUUGCUUGGUUAAAGUUCGUGAACGGUAGGUCGAUCUUG
GGGUCAUUAACGUGGA
2437-2556
(SEQ ID NO: 99)
UAAUAAAGCAAGUUCCAUUGUUCAGUUUUUAGGCGAUGUUUUUAUUU
UUAUAGGAGAGAUGUCUUUGGUACAAUUUCCUGUAUUAGGAAUUGGU
UUAAUCUUUGUAGGAACGUUGCUUGA
202-321
(SEQ ID NO: 100)
ACUAACGCUUUAUUUGUUCCUGAACAUUCGUUCAAUCCAAAGAAUAG
CUGAUCUGAAGAAUAUCUGCCUUGAGUAAGUGAAGUGUUCCAUCUUU
UCAUUGAUAUAUCAGAAAGGACAUCU
Segment 4
3165-3273
(SEQ ID NO: 101)
UUUUACCUGUACUGAAAGAUCAUGGUUUUGUUGAUAUAAGCUCGAAA
GAUGUUAAAGAGAAUAAAUUUUCUUUUGGUAGGUAUCAUGGUUUUGG
UACGGUAGAUUAUAA
Tobacco streak virus Isolate okra
RNA1
1121-1226
(SEQ ID NO: 102)
AUGGACGAAUCUUUAGUUCGCUAUGUUUCCGAAGCUGCAUUUCGACA
GUUUUCGAAGACUAAGGAACCUGAAACACUGGUUCAGUACAUAGCAA
CUAUGUAUUCUU
1035-1142
(SEQ ID NO: 103)
GCCUGAGGAAGAAAGUUUUCGUCAAAUUAGCCGUACCUGUAAGUGCC
GAAUGGUAUACUGAACAAUUCGAGGUUAGGUACGCGUUGAUGGACGA
AUCUUUAGUUCGCU
2185-2294
(SEQ ID NO: 104)
UUCUGUCAGGGUGUACGUACCUUAUGAAAAUAAGUGGUACCCCUCUG
CACCCUCCGGUCAGUACGAAAGAGCUAUGACCGUUGAUGGGUAUGUG
UCGCUUCAAUGGAAUU
3032-3130
(SEQ ID NO: 105)
AUACCGGGGAUGCCGAAGGAUAGGAUUAAAACUACCCAUGAAGCCCA
AGGUGAAACCUGGGAUCAUGUGGUGAUGUUCAGACUUUCGAAGACUA
CUAAU
RNA2
1229-1335
(SEQ ID NO: 106)
CAUAGAACGACCCAUUCCCGCUACGAUUACGUAUCAUAAGAAAGGGG
UUGUCAUGAUGACAUCCCCAUAUUUCUUAUGUGCGAUGGUGAGGUUG
CUCUAUGUGUUGA
269-378
(SEQ ID NO: 107)
GUUGAUUUCGACGGAGGUUGAUCCUUUCUACCUUCCAUACGACGAUC
UUGACGUGGACUACACCUCUUUACGUGUGUUUGGUGACGAGUACCAA
UCCUGUUCCGAUCGAG
637-743
(SEQ ID NO: 108)
GUUUCAUACCGACCUUUGAAGAAUUAAGUCGUCCGAAAUGGACACCG
AAGGUGAGUCAGGUCAAACCUGACCCUUCUGUGAUUCAGUCAGCCGU
CGAUGAACUUUUU
709-818
(SEQ ID NO: 109)
CUUCUGUGAUUCAGUCAGCCGUCGAUGAACUUUUUCCCCACCAUCAU
UCUGUCGAUGACAGGUUCUUCCAAGAAUGGGUUGAAACUCAUGAUAU
UGACUUGGAAGUCACG
2049-2158
(SEQ ID NO: 110)
AAAUGCUUGUUUCUGGAGUCUGCUUUGUUGAGUUUACCUAGUUUGGU
AGCGAAUAGAAUGAAAUUCGUUCGAAGAACUAUCAACUUAGAGAGUU
CUAAAGUUUGUAUUCG
512-582
(SEQ ID NO: 111)
UCGUGUCGUCGAUGACAUUCCUUUUGAUGACGAUGGUAAAGUCAUCG
AUGAGGUAUGGGUUGAUGCCGAGC
497-590
(SEQ ID NO: 112)
AAUGGACUUGAGCGAUCGUGUCGUCGAUGACAUUCCUUUUGAUGACG
AUGGUAAAGUCAUCGAUGAGGUAUGGGUUGAUGCCGAGCCCUCAAGG
427-534
(SEQ ID NO: 113)
CUUCUUGGGGUAGUGAGUCUGACACGUCUUUCGUUGAGCAUCUUGAA
GAAAUUCAAGGUAUACCGACGAAAAUGGACUUGAGCGAUCGUGUCGU
CGAUGACAUUCCUU
2328-2430
(SEQ ID NO: 114)
AGUGAAGGCCGAUCAGACCGACGUGAUCAAUCCAGUGGAGUUGAAAC
UGGAAGAGCGAAGCCCACCCGGAAAGGCAGGGUCAAAUUGCAUUGAU
UGCGCUAUU
1837-1946
(SEQ ID NO: 115)
UGGUUGUAGAGUGCGAUGAUGGGUCGGAAGAAGUUUUGGCAGUUCCC
AAUCCUCUGAAACUUCUCCAAAAAUUCGGUCCCAAAAACCUUCAAGU
CACCGUGUUGGAUGAU
RNA3
494-602
(SEQ ID NO: 116)
ACUUAUGCCAUCUCGGAGCUUAAAUUGAAAAAUUUAGCUACAGGUGA
UGAAUUGUAUGGUGGUACAAAAGUCGACCUGAGCAAAGCCUUCAUAU
UAACUAUGACUUGGC
375-452
(SEQ ID NO: 117)
AGUUGACUACCAAAGAGACGAAAUCCUUUAUCGGUAAAUUUUCCGAU
AAAGUUAGAGGACGUACCUUUGUAGAUCACG
1752-1858
(SEQ ID NO: 118)
AUUUUGAUCUCGGCGGUAAGCUUCUCAACCAACUAGACGAUAGAGCU
AUCGUCUGGUGCCUCGACGAAAGGCGUCGAGAUGCCAAGAGGGUUCA
GCUGGCGGGAUAU
601-710
(SEQ ID NO: 119)
GCCUCGCUCUCUAUUUGCUGAAGCAGUUCAUGCCCACAGAGGAUUGU
ACCUGGGGGGAACUGUUUCCUGCGCUUCCUCAGUGCCUUCAAACGCC
AAAAUUGGGAUGUGGU
Tobacco etch virus
7690-7798
(SEQ ID NO: 120)
GCUUUACCAAGUGGGUGGGUGUAUUGUGACGCUGAUGGUUCGCAAUU
CGACAGUUCCUUGACUCCAUUCCUCAUUAAUGCUGUAUUGAAAGUGC
GACUUGCCUUCAUGG
932-1033
(SEQ ID NO: 121)
GCUCGUACGGACCUGCGCAUUGGUAUCGACAUGGUAUGUUCAUUGUA
CGCGGUCGGUCGGAUGGGAUGUUGGUGGAUGCUCGUGCGAAGGUAAC
GUUCGCUG
3193-3302
(SEQ ID NO: 122)
GACGUCUACAAGUUUAUCACAGUCUCGAGUGUCCUUUCCUUGUUGUU
GACAUUCUUAUUUCAAAUUGACUGCAUGAUAAGGGCACACCGAGAGG
CGAAGGUUGCUGCACA
693-799
(SEQ ID NO: 123)
CCAUAUGCAGGUGGAGAUCAUUAGCAAGAAGAGCGUCCGAGCGAGGG
UCAAGAGAUUUGAGGGCUCGGUGCAAUUGUUCGCAAGUGUGCGUCAC
AUGUAUGGCGAGA
9217-9326
(SEQ ID NO: 124)
GGCAACGUGGGUACUGCAGAGGAAGACACUGAACGGCACACAGCGCA
CGAUGUGAACCGUAACAUGCACACACUAUUAGGGGUCCGCCAGUGAU
AGUUUCUGCGUGUCUU
4595-4702
(SEQ ID NO: 125)
UUGGGACUAAGGUUGUACCAGUUUUGGAUGUGGACAAUAGAGCGGUG
CAGUACAACAAAACUGUGGUGAGUUAUGGGGAGCGCAUCCAAAGACU
CGGUAGAGUUGGGC
3297-3394
(SEQ ID NO: 126)
UGCACAGUUGCAGAAAGAGAGCGAGUGGGACAAUAUCAUCAAUAGAA
CUUUCCAGUAUUCUAAGCUUGAAAAUCCUAUUGGCUAUCGCUCUACA
GCGG
4653-4738
(SEQ ID NO: 127)
AACUGUGGUGAGUUAUGGGGAGCGCAUCCAAAGACUCGGUAGAGUUG
GGCGACACAAGGAAGGAGUAGCACUUCGAAUUGGCCAAA
6953-7058
(SEQ ID NO: 128)
AACUCAUGAGUGAAUUGGUGUACUCGCAAGGGGAGAAGAGGAAAUGG
GUCGUGGAAGCACUGUCAGGGAACUUGAGGCCAGUGGCUGAGUGUCC
CAGUCAGUUAGU
8499-8606
(SEQ ID NO: 129)
UGAGAAUCUUUAUUUUCAGAGUGGCACUGUGGGUGCUGGUGUUGACG
CUGGUAAGAAGAAAGAUCAAAAGGAUGAUAAAGUCGCUGAGCAGGCU
UCAAAGGAUAGGGA
4604-4694
(SEQ ID NO: 130)
AGGUUGUACCAGUUUUGGAUGUGGACAAUAGAGCGGUGCAGUACAAC
AAAACUGUGGUGAGUUAUGGGGAGCGCAUCCAAAGACUCGGUAG
5033-5123
(SEQ ID NO: 131)
CCUCUUGGCUUACGAGUGGAGAGUAUAAGCGACUUGGUUACAUAGCA
GAGGAUGCUGGCAUAAGAAUCCCAUUCGUGUGCAAAGAAAUUCC
5229-5338
(SEQ ID NO: 132)
GCAAACGGAUGUGCACUCAAUUGCGAGGACUCUAGCAUGCAUCAAUA
GACUCAUAGCACAUGAACAAAUGAAGCAGAGUCAUUUUGAAGCCGCA
ACUGGGAGAGCAUUUU
7009-7117
(SEQ ID NO: 133)
GCACUGUCAGGGAACUUGAGGCCAGUGGCUGAGUGUCCCAGUCAGUU
AGUCACAAAGCAUGUGGUUAAAGGAAAGUGUCCCCUCUUUGAGCUCU
ACUUGCAGUUGAAUC
7617-7726
(SEQ ID NO: 134)
UGAUCUCAACAUAAAGGCACCAUGGACAGUUGGUAUGACUAAGUUUU
AUCAGGGGUGGAAUGAAUUGAUGGAGGCUUUACCAAGUGGGUGGGUG
UAUUGUGACGCUGAUG
3721-3830
(SEQ ID NO: 135)
CCUGGAGUCACUUUUAAGCAAUGGUGGAACAACCAAAUCAGCCGAGG
CAACGUGAAGCCACAUUAUAGAACUGAGGGGCACUUCAUGGAGUUUA
CCAGAGAUACUGCGGC
4701-4806
(SEQ ID NO: 136)
GCGACACAAGGAAGGAGUAGCACUUCGAAUUGGCCAAACAAAUAAAA
CACUGGUUGAAAUUCCAGAAAUGGUUGCCACUGAAGCUGCCUUUCUA
UGCUUCAUGUAC
5729-5838
(SEQ ID NO: 137)
AGGCGCGUGGGGCUAGAGGGCAAUAUGAGGUUGCAGCGGAGCCAGAG
GCGCUAGAACAUUACUUUGGAAGCGCAUAUAAUAACAAAGGAAAGCG
CAAGGGCACCACGAGA
9329-9434
(SEQ ID NO: 138)
CUUUCCGCUUUUAAGCUUAUUGUAAUAUAUAUGAAUAGCUAUUCACA
GUGGGACUUGGUCUUGUGUUGAAUGGUAUCUUAUAUGUUUUAAUAUG
UCUUAUUAGUCU

### REFERENCES

Alonso J M, et al. (2003) Genome-wide insertional mutagenesis of Arabidopsis thaliana. Science 310:653-657.
Anderson J P, et al. (2004) Antagonistic interaction between abscisic acid and jasmonate-ethylene signaling pathways modulates defence gene expression and disease resistance in Arabidopsis. The Plant Cell 16:3460-3479.
Azevedo J, et al. (2010) Argonaute quenching and global changes in Dicer homeostasis caused by a pathogen-encoded GW repeat protein. Genes Dev. 24:904-915.
Bendahmane A, Kanyuka K, Baulcombe D C (1999) The Rx gene from potato controls separate virus resistance and cell death responses. The Plant Cell 11:781-792.
Brands A, David Ho T (2002) Function of a plant stress-induced gene, HVA22. Synthetic enhancement screen with its yeast homolog reveals its role in vesicular traffic. Plant Physiology 130: 1121-1131.
Brosnan C A, et al. (2007) Nuclear gene silencing directs reception of long distance mRNA silencing in Arabidopsis. Proc Natl Acad Sci USA. 104:14741-14746.
Bruce A T, et al. (2007) ABA is essential signal for Plant resistance to pathogens affecting JA biosynthesis and the activation of defences in Arabidopsis. The Plant Cell 19:1665-1681.
Chen C, Chu C, Zentella R, Pan S, David Ho T (2002) AtHVA22 gene family in Arabidopsis: phylogenetic relationship, ABA and stress regulation, and tissue-specific expression. Plant Molecular Biology 49:633-644.
Chen N C, Chen H, Yeh S, Vittore G, David Ho T (2009) Autophagy Is enhanced and floral development is impaired in AtHVA22d RNA interference Arabidopsis. Plant Physiology 149:1679-1689.
Choy E Y, et al. (2008) An Epstein-Barr virus-encoded microRNA targets PUMA to promote host cell survival. J. Exp. Med. 205:2551-2560.
Deleris A, et al. (2006) Hierarchical action and inhibition of plant Dicer-Like proteins in antiviral defence. Science 313:68-71.
Fan J, Hill L, Crooks C, Doerner P, Lamb C (2009) Abscisic acid has a key role in modulating diverse plant-pathogen interactions. Plant Physiology 150:1750-1761.
Fusaro A F, et al. (2006) RNA interference-inducing hairpin RNAs in plants act through the viral defence pathway. EMBO Rep 7:1168-1175.
Griffiths-Jones S, Grocock R J, Dongen S V, Bateman A, Enright A J (2006) miRBase: microRNA sequences, targets and gene nomenclature. Nucleic Acids Research 34:D140-D144.
Griffiths-Jones S, Saini H K, Dongen S V, Enright A J (2008) miRBase: tools for microRNA genomics. Nucleic Acids Research 36:D154-D158.
Haasnoot J, et al. (2007) The Ebola virus VP35 protein is a suppressor of RNA silencing. PLoS Pathog. 3:e86.
Hussain M, Taft R J, Asgari S (2008) An insect virus-encoded MicroRNA regulates viral replication. Journal of Virology 82:9164-9170.
Huynh T, et al. (2006) A pattern-based method for the identification of microRNA-target sites and their corresponding RNA/RNA complexes. Cell 126:1203-1217.
Iriti M, Faoro F (2008) Abscisic acid is involved in chitosan-induced resistance to tobacco necrosisvirus (TNV). Plant Physiology and Biochemistry 46:1106-1111.
Jin-Wu N, Jinhan K, Sung-Kyu K, Byoung-Tak Z (2006) ProMiR II: a web server for the probabilistic prediction of clustered, non clustered, conserved and nonconserved microRNAs. Nucleic Acids Research 34:W455-W458.
Kasschau K D, Carrington J C (1998) A counter-defensive strategy of plant viruses: suppression of posttranscriptional gene silencing. Cell 95:461-470.
Krüger J, Rehmsmeier M (2006) RNAhybrid: microRNA target prediction easy, fast and flexible. Nucleic Acids Research 34:W451-454.
Li W X, et al. (2004) Interferon antagonist proteins of influenza and vaccinia viruses are suppressors of RNA silencing. Proc Natl Acad Sci USA. 3:1350-1355.
Li X H, Valdez P, Olvera R E, Carrington J C (1997) Functions of the tobacco etch virus RNA polymerase (NIb): subcellular transport and protein-protein interaction with VPg/proteinase (NIa). Journal of Virology 71:1598-1607.
Li Z, et al. (2009) Characterization of viral and human RNAs smaller than canonical MicroRNAs. Journal of Virology 83:12751-12758.
Lu D P, et al. (2005) PCR-based expression analysis and identification of microRNAs. J. RNAi Gene Silencing 1:44-49.
Lu S, Cullen R B (2004) Adenovirus VA1 noncoding RNA can inhibit small interfering RNA and microRNA biogenesis. Journal of Virology 78:12868-12876.
Mallory A C, Bouche N (2008) MicroRNA-directed regulation: to cleave or not to cleave. Trends in Plant Science 13:359-367.
Mlotshwa S, Pruss G J, Vance V (2008) Small RNAs in viral infection and host Defence. Trends in Plant Science 13:375-382.
Morel J B, et al. (2002) Fertile hypomorphic ARGONAUTE (ago1) mutants impaired in post-transcriptional gene silencing and virus resistance. The Plant Cell. 14: 629-639.
Murphy E, Vaníček J, Robins H, Shenk T, Levine A J (2008) Suppression of immediate-early viral gene expression by herpesvirus-coded microRNAs: Implications for latency. Proc Natl Acad Sci USA. 105:5453-5458.
Navarro B, et al. (2009) Deep sequencing of viroid-derived small RNAs from grapevine provides new insights on the role of RNA silencing in plant-viroid interaction. PLoS ONE 4:e7686.
Palatnik J F, et al. (2007) Sequence and expression differences underlie functional specialization of Arabidopsis microRNAs miR159 and miR319. Dev Cell 13:115-125.
Park M Y, Gang W, Gonzalez-Sulser A, Vaucheret H, Poething R S (2005) Nuclear processing and export of microRNAs in Arabidopsis. Proc Natl Acad Sci USA. 102:3691-3696.
Pfeffer S, et al. (2004) Identification of virus-encoded microRNAs. Science 304:734-736.
Restrepo M A, Freed D D, Carrington J C (1990) Nuclear transport of plant Potyviral proteins. The Plant Cell 2:987-998.
Schwab R, et al. (2005) Specific effects of microRNAs on the plant transcriptome. Dev. Cell 8:517-527.
Soosaar J L M, Burch-Smith T M, Dinesh-Kumar S P (2005) Mechanisms of plant resistance to viruses. Nature Reviews Microbiology 3:789-798.
Stern-Ginossar N, et al. (2007) Host immune system gene targeting by a viral miRNA. Science 317:376-381.
Tomasz T C, Meier I (2006) Isolation of nuclear proteins. Methods in Molecular Biology, 323:393-402.
Varble A, et al. (2010) Engineered RNA viral synthesis of microRNAs. Proc Natl Acad Sci USA. 107:11519-11524.
Vazquez F, Blevins T, Ailhas J, Boller T, Meins F (2008) Evolution of Arabidopsis MIR genes generates novel microRNA classes. Nucleic Acids Research 36:W6429-6438.
Wang X B, et al. (2010) RNAi-mediated viral immunity requires amplification of virus derived siRNAs in Arabidopsis thaliana. Proc Natl Acad Sci USA. 107:484-489.
Zhang Y (2005) miRU: an automated plant miRNA target prediction server. Nucleic Acids Research 33:W701-704.
Zou X, Seemann J R, Neuman D, Shen Q J (2004) A WRKY gene from creosote bush encodes an activator of the Abscisic acid signalling pathway. The Journal of Biological Chemistry 279:55770-55779.
Zuker M (2003) Mfold web server for nucleic acid folding and hybridization prediction. Nucleic Acids Research. 31:3406-3415.

What is claimed is:

1. A method of producing an isolated plant viral miRNA molecule that comprises a nucleotide sequence comprising no more than 30 contiguous nucleotides, which nucleotide sequence is capable of modulating a plant defence response, said method including the step of isolating one or more of said isolated miRNA molecules from a nucleic acid sample obtained from a plant pathogen or a plant infected with said plant pathogen.

2. The method according to claim 1, wherein said plant pathogen is a virus.

3. The method according to claim 2, wherein said plant pathogen is an RNA virus.

4. The method according to claim 3, wherein said plant pathogen is a positive sense single-stranded RNA virus.

5. The method according to claim 4, wherein said plant pathogen is a virus of the family Potyviridae or Virgaviridae.

6. The method according to claim 3, wherein said plant pathogen is a negative sense single-stranded RNA virus.

7. The method according to claim 6, wherein said plant pathogen is a virus of the family Bunyaviridae.

8. The method according to claim 3, wherein said plant pathogen is a double-stranded RNA virus.

9. The method according to claim 8, wherein said plant pathogen is a virus of the family Reoviridae.

10. A method of identifying a plant defence nucleic acid, said method including the step of identifying a plant defence nucleic acid that is modulated by an isolated plant viral miRNA molecule that comprises a nucleotide sequence comprising no more than 30 contiguous nucleotides, which nucleotide sequence is capable of modulating a plant defence response.

11. The method according to claim 10, wherein the expression and/or activity of the plant defence nucleic acid is modulated by the isolated miRNA molecule.

12. A method of modifying a plant defence nucleic acid, said method including the step of modifying a nucleotide sequence of the plant defence nucleic acid to be at least partially resistant to modulation by an isolated plant viral miRNA molecule that comprises a nucleotide sequence comprising no more than 30 contiguous nucleotides, which nucleotide sequence is capable of modulating a plant defence response.

13. A method of reducing a susceptibility of a plant population to a pathogen, said method including the step of selecting for at least one plant that comprises a naturally occurring plant defence nucleic acid that is not susceptible to modulation by an isolated plant viral miRNA molecule that comprises a nucleotide sequence comprising no more than 30 contiguous nucleotides, which nucleotide sequence is capable of modulating a plant defence response, which thereby has a reduced, decreased, or mitigated susceptibility to said pathogen, and using the at least one plant in plant breeding.

14. The method according to claim 13, wherein said pathogen is a virus.

15. The method according to claim 14, wherein said pathogen is an RNA virus.

16. The method according to claim 15, wherein said pathogen is a virus of the family Potyviridae, Virgaviridae, Bunyaviridae, or Reoviridae.

17. A method of reducing a susceptibility of a plant to a pathogen, said method including the step of introducing a decoy target sequence into said plant to thereby reduce, decrease, or mitigate the susceptibility of said plant to said pathogen, wherein said decoy target sequence binds, anneals to, hybridises to, or otherwise recognises an isolated plant viral miRNA molecule that comprises a nucleotide sequence comprising no more than 30 contiguous nucleotides, which nucleotide sequence is capable of modulating a plant defence response.

18. The method according to claim 17, wherein said pathogen is a virus.

19. The method according to claim 18, wherein said pathogen is an RNA virus.

20. The method according to claim 19, wherein said pathogen is a virus of the family Potyviridae, Virgaviridae, Bunyaviridae, or Reoviridae.


SEQUENCE LISTING (225 sequences)

SEQ ID NO: 1 - RNA, 21 nt, Turnip mosaic virus
ugcaccaucugauucagugau

SEQ ID NO: 2 - RNA, 22 nt, Turnip mosaic virus
gcgaguucccauucuaucuucu

SEQ ID NO: 3 - RNA, 21 nt, Turnip mosaic virus
guugagugcuuggugguacac

SEQ ID NO: 4 - RNA, 21 nt, Turnip mosaic virus
ugacuuugucauguguguugu

SEQ ID NO: 5 - RNA, 21 nt, Turnip mosaic virus
uaaagccuugccuguuuuguu

SEQ ID NO: 6 - RNA, 21 nt, Turnip mosaic virus
aaaacauugaucacaagagau

SEQ ID NO: 7 - RNA, 21 nt, Turnip mosaic virus
ggaaugugggugaugauggau

SEQ ID NO: 8 - RNA, 22 nt, Turnip mosaic virus
acguugggugaacacucagcaa

SEQ ID NO: 9 - RNA, 22 nt, Turnip mosaic virus
guuggugguaaagugucuagua

SEQ ID NO: 10 - RNA, 23 nt, Turnip mosaic virus
uccaaaugauuuugcugagaaau

SEQ ID NO: 11 - RNA, 22 nt, Turnip mosaic virus
caauagcgugucuuggguuggu

SEQ ID NO: 12 - RNA, 21 nt, Turnip mosaic virus
ugauggauggugacgaucagg

SEQ ID NO: 13 - RNA, 21 nt, Turnip mosaic virus
aauauaaacggaaugugggug

SEQ ID NO: 14 - RNA, 23 nt, Turnip mosaic virus
aacggaaugugggugaugaugga

SEQ ID NO: 15 - RNA, 23 nt, Turnip mosaic virus
uuuaaccgacaugagccuagcuc

SEQ ID NO: 16 - RNA, 23 nt, Turnip mosaic virus
augcauuugauuucuaugaaaug

SEQ ID NO: 17 - RNA, 22 nt, Turnip mosaic virus
auuucuaugaaaugacuucuag

SEQ ID NO: 18 - RNA, 22 nt, Turnip mosaic virus
gucgaggcuagggcuaauauca

SEQ ID NO: 19 - RNA, 21 nt, Turnip mosaic virus
auuuuauugguguuagcgcau

SEQ ID NO: 20 - RNA, 21 nt, Turnip mosaic virus
cgaaagcuauacaaccaggag

SEQ ID NO: 21 - RNA, 21 nt, Turnip mosaic virus
accaggaguaguaugugcugg

SEQ ID NO: 22 - RNA, 22 nt, Turnip mosaic virus
gcuuccuugcauaucgcaguag

SEQ ID NO: 23 - RNA, 21 nt, Turnip mosaic virus
uugcauaucgcaguagugauc

SEQ ID NO: 24 - RNA, 22 nt, Turnip mosaic virus
gaaugggucaagcacuggaagu

SEQ ID NO: 25 - RNA, 20 nt, Turnip mosaic virus
auaauaugaaggucacgaac

SEQ ID NO: 26 - RNA, 22 nt, Turnip mosaic virus
gcacaugaaugggucaagcacu

SEQ ID NO: 27 - RNA, 21 nt, Turnip mosaic virus
ugauggauggugacgaucagg

SEQ ID NO: 28 - RNA, 21 nt, Turnip mosaic virus
uggugacgaucagguggaauu

SEQ ID NO: 29 - RNA, 22 nt, Turnip mosaic virus
aucgcacgccuuuguaauuaga

SEQ ID NO: 30 - RNA, 22 nt, Turnip mosaic virus
gaaguccaucgcacgccuuugu

SEQ ID NO: 31 - RNA, 21 nt, Turnip mosaic virus
aagaauugaagaauuugacuu

SEQ ID NO: 32 - RNA, 22 nt, Turnip mosaic virus
ucaaauucuucaauucuugcuc

SEQ ID NO: 33 - RNA, 22 nt, Turnip mosaic virus
uugaagaauuugacuuuguuau

SEQ ID NO: 34 - RNA, 22 nt, Turnip mosaic virus
ucgggaauuccaccugaucguc

SEQ ID NO: 35 - RNA, 22 nt, Turnip mosaic virus
cugccuaaauguggguuuggcg

SEQ ID NO: 36 - RNA, 23 nt, Turnip mosaic virus
gguguuaaaucuaccuuuaaagc

SEQ ID NO: 37 - RNA, 21 nt, Turnip mosaic virus
aagaggcaugugugguguuaa

SEQ ID NO: 38 - RNA, 22 nt, Turnip mosaic virus
aucgaacugugauccaucugcg

SEQ ID NO: 39 - RNA, 22 nt, Turnip mosaic virus
auagugaacuaucgaacuguga

SEQ ID NO: 40 - RNA, 23 nt, Turnip mosaic virus
ccaugaauucuaaucggauguug

SEQ ID NO: 41 - RNA, 21 nt, Turnip mosaic virus
aucggauguugaguacugcgu

SEQ ID NO: 42 - RNA, 21 nt, Turnip mosaic virus
aacugugauccaucugcgucg

SEQ ID NO: 43 - RNA, 21 nt, Turnip mosaic virus
aaucaacauccaacacucgau

SEQ ID NO: 44 - RNA, 21 nt, Turnip mosaic virus
auuagcacuaugggucagaau

SEQ ID NO: 45 - RNA, 23 nt, Turnip mosaic virus
ugcgaguucccauucuaucuucu

SEQ ID NO: 46 - RNA, 22 nt, Turnip mosaic virus
aaucaacauccaacacucgaug

SEQ ID NO: 47 - RNA, 21 nt, Turnip mosaic virus
ggugagaguagggcguauagu

SEQ ID NO: 48 - RNA, 23 nt, Turnip mosaic virus
ggaaccaauuggaagucacuguu

SEQ ID NO: 49 - RNA, 22 nt, Turnip mosaic virus
auuugggaugcucugcauugag

SEQ ID NO: 50 - RNA, 21 nt, Turnip mosaic virus
uauucugcuucucuuuccuca

SEQ ID NO: 51 - RNA, 23 nt, Turnip mosaic virus
aagaagaggaaccaauuggaagu

SEQ ID NO: 52 - RNA, 21 nt, Turnip mosaic virus
gcucugcauugaggaaacuga

SEQ ID NO: 53 - RNA, 22 nt, Turnip mosaic virus
auugaggaaacugaagaagagg

SEQ ID NO: 54 - RNA, 21 nt, Turnip mosaic virus
uugcagugcuugcgguucgag

SEQ ID NO: 55 - RNA, 22 nt, Turnip mosaic virus
ugcguguaccuguggaugcauu

SEQ ID NO: 56 - RNA, 22 nt, Turnip mosaic virus
uaucucaccacuugacuugugu

SEQ ID NO: 57 - RNA, 23 nt, Turnip mosaic virus
cgugugcucucgaucacuacugc

SEQ ID NO: 58 - RNA, 21 nt, Turnip mosaic virus
ggaagcaccuaucaaagccuu

SEQ ID NO: 59 - RNA, 22 nt, Turnip mosaic virus
gguggugguguuggugauagcu

SEQ ID NO: 60 - RNA, 22 nt, Turnip mosaic virus
agugcugguuuguugguggugg

SEQ ID NO: 61 - RNA, 23 nt, Turnip mosaic virus
ggugauaaacacacacuucagua

SEQ ID NO: 62 - RNA, 21 nt, Turnip mosaic virus
auguugaguacugcguugauu

SEQ ID NO: 63 - RNA, 22 nt, Turnip mosaic virus
aucgaacugugauccaucugcg

SEQ ID NO: 64 - RNA, 22 nt, Turnip mosaic virus
auagugaacuaucgaacuguga

SEQ ID NO: 65 - RNA, 23 nt, Turnip mosaic virus
auccaucugcgucgcaguaaauc

SEQ ID NO: 66 - RNA, 22 nt, Turnip mosaic virus
guuggugguaaagugucuagua

SEQ ID NO: 67 - RNA, 21 nt, Turnip mosaic virus
aauauaaacggaaugugggug

SEQ ID NO: 68 - RNA, 22 nt, Turnip mosaic virus
gcuuuuccaaaugauuuugcug

SEQ ID NO: 69 - RNA, 21 nt, Turnip mosaic virus
ucgccauauuuaaucaacgca

SEQ ID NO: 70 - RNA, 20 nt, Turnip mosaic virus
acagagcaagaauugaagaa

SEQ ID NO: 71 - RNA, 21 nt, Turnip mosaic virus
aauauaacaaagucaaauucu

SEQ ID NO: 72 - RNA, 21 nt, Turnip mosaic virus
aguggaacaaaacauugauca

SEQ ID NO: 73 - RNA, 22 nt, Turnip mosaic virus
ggaguucuaggagguggaauuu

SEQ ID NO: 74 - RNA, 21 nt, Turnip mosaic virus
auuccaccuccuagaacucca

SEQ ID NO: 75 - RNA, 22 nt, Turnip mosaic virus
uagacaccauggcagacaauuu

SEQ ID NO: 76 - RNA, 22 nt, Turnip mosaic virus
auuagauucuuuguuaauggcg

SEQ ID NO: 77 - RNA, 22 nt, Turnip mosaic virus
cuuuguuaauggcgaugaucug

SEQ ID NO: 78 - RNA, 21 nt, Turnip mosaic virus
auggguagagaaguuuauggg

SEQ ID NO: 79 - RNA, 22 nt, Turnip mosaic virus
ugacgacaccauagaacacuuc

SEQ ID NO: 80 - RNA, 21 nt, Turnip mosaic virus
gagaaguuuauggggaugacg

SEQ ID NO: 81 - RNA, 22 nt, Turnip mosaic virus
gcuugcagucucccaaaacuga

SEQ ID NO: 82 - RNA, 21 nt, Turnip mosaic virus
ucgucuucauagucuucgaau

SEQ ID NO: 83 - DNA, 22 bp, Artificial Sequence
  qRT-PCR Primer Beta-Actin 7-Reverse
gaggaagagcattcccctcgta

SEQ ID NO: 84 - DNA, 24 bp, Artificial Sequence
  qRT-PCR primer Beta-Actin 2-Reverse
gatggcatggaggaagagagaaac

SEQ ID NO: 85 - DNA, 23 bp, Artificial Sequence
  qRT-PCR Universal Actin forward primer
agtggtcgtacaaccggtattgt

SEQ ID NO: 86 - DNA, 25 bp, Artificial Sequence
  qRT-PCR Primer GFP forward
aaccattacctgtccacacaatctg

SEQ ID NO: 87 - DNA, 26 bp, Artificial Sequence
  qRT-PCR primer GFP reverse
atagttcatccatgccatgtgtaatc

SEQ ID NO: 88 - DNA, 21 bp, Arabidopsis thaliana
ctcacagtcactgaatcagaa

SEQ ID NO: 89 - DNA, 21 bp, Artificial Sequence
  Mutated sequence
ctcacagccattagccacata

SEQ ID NO: 90 - DNA, 21 bp, Artificial Sequence
  Example of a decoy sequence to capture TuMV miRNA
atcactgaatcagatggtgca

SEQ ID NO: 91 - RNA, 120 nt, Fiji disease virus
aacuuaauuuuaaacgcaccaccuuaucuugguguuuaacaaauucuacaaacuuaaacauguguucuucaucgcuaguaaaaaacaucugauugauaagcaagaaagcgccaucuccau

SEQ ID NO: 92 - RNA, 110 nt, Fiji disease virus
cuaccuaauuuaacuuuguuuuuacgacguaaaaugauugaaguauuauaugaacuugacccuacaggcaaaugugcuagauauuucuucgcugaagauaaagaauacug

SEQ ID NO: 93 - RNA, 120 nt, Fiji disease virus
uuaguucuuuagucaaagguucuguuccgaugaauaaaucccggaucuuuucugcuuuauugaaaauggcaucaaucauguuaacuuuaucauuaagaauuuuuucauacucagcaacuu

SEQ ID NO: 94 - RNA, 107 nt, Fiji disease virus
gaauagucacaaauuugguaaacauuuguaugauuuaaugucaguauuuuguaggucagaacugauagcguaugaagccagguauggauguuuuauuaaauuugaga

SEQ ID NO: 95 - RNA, 120 nt, Fiji disease virus
gauugguuuguggauacuuuacuaguaguucgaauuccccugaugacuauuuuaguguugaugaagauacuuuauauuucaguauugauuuggaugaacauccugaaguguuuacgaccg

SEQ ID NO: 96 - RNA, 110 nt, Fiji disease virus
guguugaugaagauacuuuauauuucaguauugauuuggaugaacauccugaaguguuuacgaccguuggcacaaauggauucaguauacaguuacaauuuaagaaagga

SEQ ID NO: 97 - RNA, 120 nt, Fiji disease virus
augcauuaauauuccuaaauuauuagcgguguuaccgauaguaucuaagauuagaucauaugcuucuuuaguugauguuuuaguagguuuaugaguaaaauaucucuggcuuagaucuau

SEQ ID NO: 98 - RNA, 110 nt, Fiji disease virus
aauccguguuaaagacaauucuagcuuucugagauuaugaagcuauguuuuggagauugcuugguuaaaguucgugaacgguaggucgaucuuggggucauuaacgugga

SEQ ID NO: 99 - RNA, 120 nt, Fiji disease virus
uaauaaagcaaguuccauuguucaguuuuuaggcgauguuuuuauuuuuauaggagagaugucuuugguacaauuuccuguauuaggaauugguuuaaucuuuguaggaacguugcuuga

SEQ ID NO: 100 - RNA, 120 nt, Fiji disease virus
acuaacgcuuuauuuguuccugaacauucguucaauccaaagaauagcugaucugaagaauaucugccuugaguaagugaaguguuccaucuuuucauugauauaucagaaaggacaucu

SEQ ID NO: 101 - RNA, 109 nt, Fiji disease virus
uuuuaccuguacugaaagaucaugguuuuguugauauaagcucgaaagauguuaaagagaauaaauuuucuuuugguagguaucaugguuuugguacgguagauuauaa

SEQ ID NO: 102 - RNA, 106 nt, Tobacco streak virus
auggacgaaucuuuaguucgcuauguuuccgaagcugcauuucgacaguuuucgaagacuaaggaaccugaaacacugguucaguacauagcaacuauguauucuu

SEQ ID NO: 103 - RNA, 108 nt, Tobacco streak virus
gccugaggaagaaaguuuucgucaaauuagccguaccuguaagugccgaaugguauacugaacaauucgagguuagguacgcguugauggacgaaucuuuaguucgcu

SEQ ID NO: 104 - RNA, 110 nt, Tobacco streak virus
uucugucaggguguacguaccuuaugaaaauaagugguaccccucugcacccuccggucaguacgaaagagcuaugaccguugauggguaugugucgcuucaauggaauu

SEQ ID NO: 105 - RNA, 99 nt, Tobacco streak virus
auaccggggaugccgaaggauaggauuaaaacuacccaugaagcccaaggugaaaccugggaucauguggugauguucagacuuucgaagacuacuaau

SEQ ID NO: 106 - RNA, 107 nt, Tobacco streak virus
cauagaacgacccauucccgcuacgauuacguaucauaagaaagggguugucaugaugacauccccauauuucuuaugugcgauggugagguugcucuauguguuga

SEQ ID NO: 107 - RNA, 110 nt, Tobacco streak virus
guugauuucgacggagguugauccuuucuaccuuccauacgacgaucuugacguggacuacaccucuuuacguguguuuggugacgaguaccaauccuguuccgaucgag

SEQ ID NO: 108 - RNA, 107 nt, Tobacco streak virus
guuucauaccgaccuuugaagaauuaagucguccgaaauggacaccgaaggugagucaggucaaaccugacccuucugugauucagucagccgucgaugaacuuuuu

SEQ ID NO: 109 - RNA, 110 nt, Tobacco streak virus
cuucugugauucagucagccgucgaugaacuuuuuccccaccaucauucugucgaugacagguucuuccaagaauggguugaaacucaugauauugacuuggaagucacg

SEQ ID NO: 110 - RNA, 110 nt, Tobacco streak virus
aaaugcuuguuucuggagucugcuuuguugaguuuaccuaguuugguagcgaauagaaugaaauucguucgaagaacuaucaacuuagagaguucuaaaguuuguauucg

SEQ ID NO: 111 - RNA, 71 nt, Tobacco streak virus
ucgugucgucgaugacauuccuuuugaugacgaugguaaagucaucgaugagguauggguugaugccgagc

SEQ ID NO: 112 - RNA, 94 nt, Tobacco streak virus
aauggacuugagcgaucgugucgucgaugacauuccuuuugaugacgaugguaaagucaucgaugagguauggguugaugccgagcccucaagg

SEQ ID NO: 113 - RNA, 108 nt, Tobacco streak virus
cuucuugggguagugagucugacacgucuuucguugagcaucuugaagaaauucaagguauaccgacgaaaauggacuugagcgaucgugucgucgaugacauuccuu

SEQ ID NO: 114 - RNA, 103 nt, Tobacco streak virus
agugaaggccgaucagaccgacgugaucaauccaguggaguugaaacuggaagagcgaagcccacccggaaaggcagggucaaauugcauugauugcgcuauu

SEQ ID NO: 115 - RNA, 110 nt, Tobacco streak virus
ugguuguagagugcgaugaugggucggaagaaguuuuggcaguucccaauccucugaaacuucuccaaaaauucggucccaaaaaccuucaagucaccguguuggaugau

SEQ ID NO: 116 - RNA, 109 nt, Tobacco streak virus
acuuaugccaucucggagcuuaaauugaaaaauuuagcuacaggugaugaauuguauggugguacaaaagucgaccugagcaaagccuucauauuaacuaugacuuggc

SEQ ID NO: 117 - RNA, 78 nt, Tobacco streak virus
aguugacuaccaaagagacgaaauccuuuaucgguaaauuuuccgauaaaguuagaggacguaccuuuguagaucacg

SEQ ID NO: 118 - RNA, 107 nt, Tobacco streak virus
auuuugaucucggcgguaagcuucucaaccaacuagacgauagagcuaucgucuggugccucgacgaaaggcgucgagaugccaagaggguucagcuggcgggauau

SEQ ID NO: 119 - RNA, 110 nt, Tobacco streak virus
gccucgcucucuauuugcugaagcaguucaugcccacagaggauuguaccuggggggaacuguuuccugcgcuuccucagugccuucaaacgccaaaauugggauguggu

SEQ ID NO: 120 - RNA, 109 nt, Tobacco etch virus
gcuuuaccaaguggguggguguauugugacgcugaugguucgcaauucgacaguuccuugacuccauuccucauuaaugcuguauugaaagugcgacuugccuucaugg

SEQ ID NO: 121 - RNA, 102 nt, Tobacco etch virus
gcucguacggaccugcgcauugguaucgacaugguauguucauuguacgcggucggucggaugggauguugguggaugcucgugcgaagguaacguucgcug

SEQ ID NO: 122 - RNA, 110 nt, Tobacco etch virus
gacgucuacaaguuuaucacagucucgaguguccuuuccuuguuguugacauucuuauuucaaauugacugcaugauaagggcacaccgagaggcgaagguugcugcaca

SEQ ID NO: 123 - RNA, 107 nt, Tobacco etch virus
ccauaugcagguggagaucauuagcaagaagagcguccgagcgagggucaagagauuugagggcucggugcaauuguucgcaagugugcgucacauguauggcgaga

SEQ ID NO: 124 - RNA, 110 nt, Tobacco etch virus
ggcaacguggguacugcagaggaagacacugaacggcacacagcgcacgaugugaaccguaacaugcacacacuauuagggguccgccagugauaguuucugcgugucuu

SEQ ID NO: 125 - RNA, 108 nt, Tobacco etch virus
uugggacuaagguuguaccaguuuuggauguggacaauagagcggugcaguacaacaaaacuguggugaguuauggggagcgcauccaaagacucgguagaguugggc

SEQ ID NO: 126 - RNA, 98 nt, Tobacco etch virus
ugcacaguugcagaaagagagcgagugggacaauaucaucaauagaacuuuccaguauucuaagcuugaaaauccuauuggcuaucgcucuacagcgg

SEQ ID NO: 127 - RNA, 86 nt, Tobacco etch virus
aacuguggugaguuauggggagcgcauccaaagacucgguagaguugggcgacacaaggaaggaguagcacuucgaauuggccaaa

SEQ ID NO: 128 - RNA, 106 nt, Tobacco etch virus
aacucaugagugaauugguguacucgcaaggggagaagaggaaaugggucguggaagcacugucagggaacuugaggccaguggcugagugucccagucaguuagu

SEQ ID NO: 129 - RNA, 108 nt, Tobacco etch virus
ugagaaucuuuauuuucagaguggcacugugggugcugguguugacgcugguaagaagaaagaucaaaaggaugauaaagucgcugagcaggcuucaaaggauaggga

SEQ ID NO: 130 - RNA, 91 nt, Tobacco etch virus
agguuguaccaguuuuggauguggacaauagagcggugcaguacaacaaaacuguggugaguuauggggagcgcauccaaagacucgguag

SEQ ID NO: 131 - RNA, 91 nt, Tobacco etch virus
ccucuuggcuuacgaguggagaguauaagcgacuugguuacauagcagaggaugcuggcauaagaaucccauucgugugcaaagaaauucc

SEQ ID NO: 132 - RNA, 110 nt, Tobacco etch virus
gcaaacggaugugcacucaauugcgaggacucuagcaugcaucaauagacucauagcacaugaacaaaugaagcagagucauuuugaagccgcaacugggagagcauuuu

SEQ ID NO: 133 - RNA, 109 nt, Tobacco etch virus
gcacugucagggaacuugaggccaguggcugagugucccagucaguuagucacaaagcaugugguuaaaggaaaguguccccucuuugagcucuacuugcaguugaauc

SEQ ID NO: 134 - RNA, 110 nt, Tobacco etch virus
ugaucucaacauaaaggcaccauggacaguugguaugacuaaguuuuaucagggguggaaugaauugauggaggcuuuaccaaguggguggguguauugugacgcugaug

SEQ ID NO: 135 - RNA, 110 nt, Tobacco etch virus
ccuggagucacuuuuaagcaaugguggaacaaccaaaucagccgaggcaacgugaagccacauuauagaacugaggggcacuucauggaguuuaccagagauacugcggc

SEQ ID NO: 136 - RNA, 106 nt, Tobacco etch virus
gcgacacaaggaaggaguagcacuucgaauuggccaaacaaauaaaacacugguugaaauuccagaaaugguugccacugaagcugccuuucuaugcuucauguac

SEQ ID NO: 137 - RNA, 110 nt, Tobacco etch virus
aggcgcguggggcuagagggcaauaugagguugcagcggagccagaggcgcuagaacauuacuuuggaagcgcauauaauaacaaaggaaagcgcaagggcaccacgaga

SEQ ID NO: 138 - RNA, 106 nt, Tobacco etch virus
cuuuccgcuuuuaagcuuauuguaauauauaugaauagcuauucacagugggacuuggucuuguguugaaugguaucuuauauguuuuaauaugucuuauuagucu

SEQ ID NO: 139 - RNA, 21 nt, Turnip mosaic virus
aucacugaaucagauggugca

SEQ ID NO: 140 - RNA, 22 nt, Turnip mosaic virus
agaagauagaaugggaacucgc

SEQ ID NO: 141 - RNA, 21 nt, Turnip mosaic virus
guguaccaccaagcacucaac

SEQ ID NO: 142 - RNA, 21 nt, Turnip mosaic virus
acaacacacaugacaaaguca

SEQ ID NO: 143 - RNA, 21 nt, Turnip mosaic virus
aacaaaacaggcaaggcuuua

SEQ ID NO: 144 - RNA, 21 nt, Turnip mosaic virus
aucucuugugaucaauguuuu

SEQ ID NO: 145 - RNA, 21 nt, Turnip mosaic virus
auccaucaucacccacauucc

SEQ ID NO: 146 - RNA, 22 nt, Turnip mosaic virus
uugcugaguguucacccaacgu

SEQ ID NO: 147 - RNA, 22 nt, Turnip mosaic virus
uacuagacacuuuaccaccaac

SEQ ID NO: 148 - RNA, 23 nt, Turnip mosaic virus
auuucucagcaaaaucauuugga

SEQ ID NO: 149 - RNA, 22 nt, Turnip mosaic virus
accaacccaagacacgcuauug

SEQ ID NO: 150 - RNA, 21 nt, Turnip mosaic virus
ccugaucgucaccauccauca

SEQ ID NO: 151 - RNA, 21 nt, Turnip mosaic virus
cacccacauuccguuuauauu

SEQ ID NO: 152 - RNA, 23 nt, Turnip mosaic virus
uccaucaucacccacauuccguu

SEQ ID NO: 153 - RNA, 23 nt, Turnip mosaic virus
gagcuaggcucaugucgguuaaa

SEQ ID NO: 154 - RNA, 23 nt, Turnip mosaic virus
cauuucauagaaaucaaaugcau

SEQ ID NO: 155 - RNA, 22 nt, Turnip mosaic virus
cuagaagucauuucauagaaau

SEQ ID NO: 156 - RNA, 22 nt, Turnip mosaic virus
ugauauuagcccuagccucgac

SEQ ID NO: 157 - RNA, 21 nt, Turnip mosaic virus
augcgcuaacaccaauaaaau

SEQ ID NO: 158 - RNA, 21 nt, Turnip mosaic virus
cuccugguuguauagcuuucg

SEQ ID NO: 159 - RNA, 21 nt, Turnip mosaic virus
ccagcacauacuacuccuggu

SEQ ID NO: 160 - RNA, 22 nt, Turnip mosaic virus
cuacugcgauaugcaaggaagc

SEQ ID NO: 161 - RNA, 21 nt, Turnip mosaic virus
gaucacuacugcgauaugcaa

SEQ ID NO: 162 - RNA, 22 nt, Turnip mosaic virus
acuuccagugcuugacccauuc

SEQ ID NO: 163 - RNA, 20 nt, Turnip mosaic virus
guucgugaccuucauauuau

SEQ ID NO: 164 - RNA, 22 nt, Turnip mosaic virus
agugcuugacccauucaugugc

SEQ ID NO: 165 - RNA, 21 nt, Turnip mosaic virus
ccugaucgucaccauccauca

SEQ ID NO: 166 - RNA, 21 nt, Turnip mosaic virus
aauuccaccugaucgucacca

SEQ ID NO: 167 - RNA, 22 nt, Turnip mosaic virus
ucuaauuacaaaggcgugcgau

SEQ ID NO: 168 - RNA, 22 nt, Turnip mosaic virus
acaaaggcgugcgauggacuuc

SEQ ID NO: 169 - RNA, 21 nt, Turnip mosaic virus
aagucaaauucuucaauucuu

SEQ ID NO: 170 - RNA, 22 nt, Turnip mosaic virus
gagcaagaauugaagaauuuga

SEQ ID NO: 171 - RNA, 22 nt, Turnip mosaic virus
auaacaaagucaaauucuucaa

SEQ ID NO: 172 - RNA, 22 nt, Turnip mosaic virus
gacgaucagguggaauucccga

SEQ ID NO: 173 - RNA, 22 nt, Turnip mosaic virus
cgccaaacccacauuuaggcag

SEQ ID NO: 174 - RNA, 23 nt, Turnip mosaic virus
gcuuuaaagguagauuuaacacc

SEQ ID NO: 175 - RNA, 21 nt, Turnip mosaic virus
uuaacaccacacaugccucuu

SEQ ID NO: 176 - RNA, 22 nt, Turnip mosaic virus
cgcagauggaucacaguucgau

SEQ ID NO: 177 - RNA, 22 nt, Turnip mosaic virus
ucacaguucgauaguucacuau

SEQ ID NO: 178 - RNA, 23 nt, Turnip mosaic virus
caacauccgauuagaauucaugg

SEQ ID NO: 179 - RNA, 21 nt, Turnip mosaic virus
acgcaguacucaacauccgau

SEQ ID NO: 180 - RNA, 21 nt, Turnip mosaic virus
cgacgcagauggaucacaguu

SEQ ID NO: 181 - RNA, 21 nt, Turnip mosaic virus
aucgaguguuggauguugauu

SEQ ID NO: 182 - RNA, 21 nt, Turnip mosaic virus
auucugacccauagugcuaau

SEQ ID NO: 183 - RNA, 23 nt, Turnip mosaic virus
agaagauagaaugggaacucgca

SEQ ID NO: 184 - RNA, 22 nt, Turnip mosaic virus
caucgaguguuggauguugauu

SEQ ID NO: 185 - RNA, 21 nt, Turnip mosaic virus
acuauacgcccuacucucacc

SEQ ID NO: 186 - RNA, 23 nt, Turnip mosaic virus
aacagugacuuccaauugguucc

SEQ ID NO: 187 - RNA, 22 nt, Turnip mosaic virus
cucaaugcagagcaucccaaau

SEQ ID NO: 188 - RNA, 21 nt, Turnip mosaic virus
ugaggaaagagaagcagaaua

SEQ ID NO: 189 - RNA, 23 nt, Turnip mosaic virus
acuuccaauugguuccucuucuu

SEQ ID NO: 190 - RNA, 21 nt, Turnip mosaic virus
ucaguuuccucaaugcagagc

SEQ ID NO: 191 - RNA, 22 nt, Turnip mosaic virus
ccucuucuucaguuuccucaau

SEQ ID NO: 192 - RNA, 21 nt, Turnip mosaic virus
cucgaaccgcaagcacugcaa

SEQ ID NO: 193 - RNA, 22 nt, Turnip mosaic virus
aaugcauccacagguacacgca

SEQ ID NO: 194 - RNA, 22 nt, Turnip mosaic virus
acacaagucaaguggugagaua

SEQ ID NO: 195 - RNA, 23 nt, Turnip mosaic virus
gcaguagugaucgagagcacacg

SEQ ID NO: 196 - RNA, 21 nt, Turnip mosaic virus
aaggcuuugauaggugcuucc

SEQ ID NO: 197 - RNA, 22 nt, Turnip mosaic virus
agcuaucaccaacaccaccacc

SEQ ID NO: 198 - RNA, 22 nt, Turnip mosaic virus
ccaccaccaacaaaccagcacu

SEQ ID NO: 199 - RNA, 23 nt, Turnip mosaic virus
uacugaaguguguguuuaucacc

SEQ ID NO: 200 - RNA, 21 nt, Turnip mosaic virus
aaucaacgcaguacucaacau

SEQ ID NO: 201 - RNA, 22 nt, Turnip mosaic virus
cgcagauggaucacaguucgau

SEQ ID NO: 202 - RNA, 22 nt, Turnip mosaic virus
ucacaguucgauaguucacuau

SEQ ID NO: 203 - RNA, 23 nt, Turnip mosaic virus
gauuuacugcgacgcagauggau

SEQ ID NO: 204 - RNA, 22 nt, Turnip mosaic virus
uacuagacacuuuaccaccaac

SEQ ID NO: 205 - RNA, 21 nt, Turnip mosaic virus
cacccacauuccguuuauauu

SEQ ID NO: 206 - RNA, 22 nt, Turnip mosaic virus
cagcaaaaucauuuggaaaagc

SEQ ID NO: 207 - RNA, 21 nt, Turnip mosaic virus
ugcguugauuaaauauggcga

SEQ ID NO: 208 - RNA, 21 nt, Turnip mosaic virus
uucuucaauucuugcucuguu

SEQ ID NO: 209 - RNA, 21 nt, Turnip mosaic virus
agaauuugacuuuguuauauu

SEQ ID NO: 210 - RNA, 21 nt, Turnip mosaic virus
ugaucaauguuuuguuccacu

SEQ ID NO: 211 - RNA, 22 nt, Turnip mosaic virus
aaauuccaccuccuagaacucc

SEQ ID NO: 212 - RNA, 21 nt, Turnip mosaic virus
uggaguucuaggagguggaau

SEQ ID NO: 213 - RNA, 22 nt, Turnip mosaic virus
aaauugucugccauggugucua

SEQ ID NO: 214 - RNA, 22 nt, Turnip mosaic virus
cgccauuaacaaagaaucuaau

SEQ ID NO: 215 - RNA, 22 nt, Turnip mosaic virus
cagaucaucgccauuaacaaag

SEQ ID NO: 216 - RNA, 21 nt, Turnip mosaic virus
cccauaaacuucucuacccau

SEQ ID NO: 217 - RNA, 22 nt, Turnip mosaic virus
gaaguguucuauggugucguca

SEQ ID NO: 218 - RNA, 21 nt, Turnip mosaic virus
cgucauccccauaaacuucuc

SEQ ID NO: 219 - RNA, 22 nt, Turnip mosaic virus
ucaguuuugggagacugcaagc

SEQ ID NO: 220 - RNA, 21 nt, Turnip mosaic virus
auucgaagacuaugaagacga

SEQ ID NO: 221 - RNA, 26 nt, Turnip mosaic virus
aauaaguauacagccuugguuuacua

SEQ ID NO: 222 - RNA, 21 nt, Turnip mosaic virus
ugacuuuccuaaccacaggug

SEQ ID NO: 223 - RNA, 25 nt, Arabidopsis plant
gucacugaaucagaaggguggagca

SEQ ID NO: 224 - RNA, 22 nt, Arabidopsis plant
gucacugaaucagaagggugga

SEQ ID NO: 225 - RNA, 21 nt, Turnip mosaic virus
uagugacuuagucuaccacgu